(19) United States
(12) Patent Application Publication
(10) Pub. No.: US 2015/0119652 A1
(43) Pub. Date: Apr. 30, 2015

(54) TELEMEDICINE VISUAL MONITORING DEVICE WITH STRUCTURED ILLUMINATION

(71) Applicant: Elwha LLC, Bellevue, WA (US)

(72) Inventors: Roderick A. Hyde, Redmond, WA (US); Jordin T. Kare, Seattle, WA (US); Elizabeth A. Sweeney, Seattle, WA (US); Lowell L. Wood, JR., Bellevue, WA (US)

(73) Assignee: Elwha LLC, a limited liability company of the State of Delaware

(21) Appl. No.: 14/068,188

(22) Filed: Oct. 31, 2013

(51) Int. Cl. A61B 5/00; A61M 35/00; A61M 5/172

(52) U.S. Cl. CPC A61B 5/0022; A61B 5/0064; A61M 5/1723; A61M 35/00
USPC 600/301; 600/476; 600/473; 604/65; 604/290

(57) ABSTRACT

A system for providing telemedicine support with structured illumination and related methods is described. The telemedicine system provides two-way audio visual communication between a patient and a remote caregiver, as well as controllable illumination of the patient of that can be remotely adjusted to enhance medically-relevant information that can be derived from images of the patient. In various aspects, lighting system parameters including but not limited to intensity, light pulse duration, spectral content, divergence or convergence and polarization can be adjusted. In some aspects, parameters of the imaging system such as filtration, pan, tilt, or zoom of the imaging system can be adjusted. In addition to providing two-way communication and controllable illumination capability, telemedicine systems as described herein may include various diagnostic or medical treatment devices.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### RELATED APPLICATIONS

U.S. patent application Ser. No. 13/930,928, entitled MEDICAL SUPPORT SYSTEM INCLUDING MEDICAL EQUIPMENT CASE, naming RODERICK A. HYDE, JORDIN T. KARE, ELIZABETH A. SWEENEY, AND LOWELL L. WOOD, JR. as inventors, filed 28 Jun. 2013 with Attorney Docket No. 0712-004-001-000000 is related to the present application.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### PRIORITY APPLICATIONS

None.

### SUMMARY

In one aspect, a method of providing remote visualization of a subject includes, but is not limited to, acquiring a first image of at least a portion of a subject at a first location at a first lighting condition with an imaging system located at the first location under control of electrical control circuitry; transmitting the acquired first image to a second location remote from the first location under control of the electrical control circuitry; receiving a lighting control signal for controlling an adjustment to a lighting condition at the first location from the second location under control of the electrical control circuitry; adjusting at least one controllable light source at the first location under control of the electrical control circuitry to provide a second lighting condition responsive to receiving the lighting control signal for controlling the adjustment to the lighting condition at the first location; acquiring a second image of the at least a portion of the subject at the first location at the second lighting condition with the imaging system located at the first location under control of the electrical control circuitry; and transmitting the acquired second image to the second location under control of the electrical control circuitry; wherein at least one of the first image and the second image contains information indicative of a health status of the subject. In addition to the foregoing, other method aspects are described herein in the claims, drawings, and text forming a part of the disclosure set forth herein.

In one aspect, a remote visualization system includes, but is not limited to an audio input device at a first location, an imaging system at the first location adapted to acquire an image of a subject containing information indicative of a health status of the subject, a video output device at the first location, an audio output device at the first location, a controllable lighting system including at least one light source adapted to illuminate at least a portion of the subject during acquisition of the image of the subject, the image containing information indicative of the health status of the subject and having at least one controllable parameter that influences at least one of the amount or type of information indicative of the health status of the subject in an acquired image of the subject, electrical control circuitry at the first location operatively connected to and configured to control operation of the audio input device, imaging system, video output device, audio output device, and controllable lighting system, and communication circuitry at the first location configured to provide communication between the electrical control circuitry and at least one electrical control circuitry at a second location that is remote from the first location. In addition to the foregoing, other system aspects are described herein in the claims, drawings, and text forming a part of the disclosure set forth herein.

In one aspect, a method of providing remote visualization of a subject includes, but is not limited to providing a subject with a remote visualization system in a transport container, the remote visualization system including an audio input device, an imaging system, a video output device, an audio output device, a controllable lighting system including at least one light source, the controllable lighting system built into or received in the container, the electrical control circuitry built into or received in the container, the electrical control circuitry configured to control operation of the audio input device, imaging system, video output device, audio output device, and controllable lighting system, and communication circuitry configured to provide communication between the electrical control circuitry at a first location and remote electrical control circuitry at a second location remote from the first location; receiving at the second location a first image of at least a portion of a subject via the communication circuitry, wherein the first image was captured at a first lighting condition with the imaging system located at the first location; transmitting a lighting control signal from the second location to the first location via the communication circuitry for controlling an adjustment to the controllable lighting system to provide a second lighting condition at the first location; and receiving at the second location a second image of the at least a portion of the subject via the communication circuitry, wherein the second image was captured at the second lighting condition with the imaging system at the first location; wherein at least one of the first image and the second image contains information indicative of a health status of the subject, and wherein the adjustment to the controllable lighting system influences at least one of the amount or type of information indicative of the health status of the subject in the second image of the subject. In addition to the foregoing, other method aspects are described herein in the claims, drawings, and text forming a part of the disclosure set forth herein.

In one aspect, an article of manufacture includes one or more non-transitory machine-readable data storage media bearing one or more instructions for providing a subject with a remote visualization system in a transport container, the remote visualization system including an audio input device, an imaging system, a video output device, an audio output device, a controllable lighting system including at least one light source, the controllable lighting system built into or received in the container, electrical control circuitry built into or received in the container, the electrical control circuitry configured to control operation of the audio input device, imaging system, video output device, audio output device, and controllable lighting system, and communication circuitry configured to provide communication between the electrical control circuitry at a first location and remote electrical control circuitry at a second location remote from the first location; receiving at the second location a first image of at least a portion of a subject via the communication circuitry, wherein the first image was captured at a first lighting condition with the imaging system located at the first location; transmitting a lighting control signal from the second location to the first location via the communication circuitry for controlling an adjustment to the controllable lighting system to provide a second lighting condition at the first location; and receiving at the second location a second image of the at least a portion of the subject via the communication circuitry, wherein the second image was captured at the second lighting condition with the imaging system at the first location; wherein at least one of the first image and the second image contains information indicative of a health status of the subject, and wherein the adjustment to the controllable lighting system influences at least one of the amount or type of information indicative of the health status of the subject in the second image of the subject. In addition to the foregoing, other aspects of articles of manufacture including one or more non-transitory machine-readable data storage media bearing one or more instructions are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is an illustration of an embodiment of a remote visualization system.

FIG. 2 is block diagram of a remote visualization system.

FIG. 3 illustrates a remote visualization system configured in part as a hand-held unit.

FIG. 4 illustrates a remote visualization system configured in part as a mobile robot.

FIG. 5 illustrates a remote visualization system in a transport container.

FIG. 6 is a flow diagram of a method of providing remote visualization of a subject.

FIG. 7 is a flow diagram of a method of providing remote visualization of a subject.

FIG. 8 is a flow diagram of a method of providing remote visualization of a subject.

FIG. 9 is a flow diagram of a method of providing remote visualization of a subject.

FIG. 10 is a flow diagram of a method of providing remote visualization of a subject.

FIG. 11 is a flow diagram of a method of providing remote visualization of a subject.

FIG. 12 is a flow diagram of a method of providing remote visualization of a subject.

FIG. 13 is a flow diagram of a method of providing remote visualization of a subject.

FIG. 14 is a flow diagram of a method of providing remote visualization of a subject.

FIG. 15 is a flow diagram of a method of providing remote visualization of a subject.

FIG. 16 is a flow diagram of a method of providing remote visualization of a subject.

FIG. 17 is a flow diagram of a method of providing remote visualization of a subject.

FIG. 18 is a flow diagram of a method of providing remote visualization of a subject.

FIG. 19 illustrates an article of manufacture including non-transitory machine readable data storage media bearing one or more instructions.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

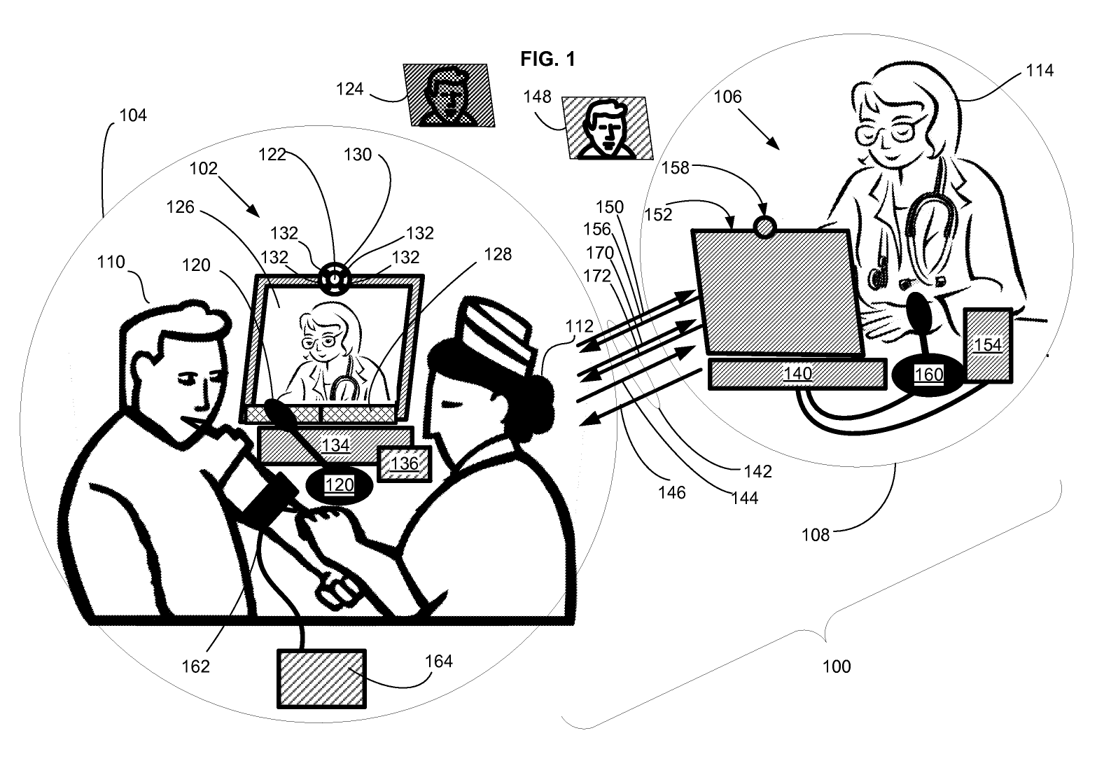

FIG. 1 depicts an example of a telemedicine system 100 for use in telemedicine applications. Telemedicine methods and systems are used, for example, in situations where a patient needs or would benefit from medical monitoring, treatment and consultation with a doctor, nurse, or other medical caregiver but is unable to safely or conveniently travel to a medical care facility for the monitoring, treatment, or consultation, or in situations where it is preferable for the patient to stay at home rather than stay at a hospital, or other facility at which medical monitoring, treatment or consultation would typically be provided. Use of telemedicine systems allows medical care to be provided in remote locations where medical caregivers are unavailable, and allows patients to receive medical monitoring, treatment, and consultation from home or another suitable location rather than in the hospital. Thus, patients may be discharged from the hospital sooner after treatment, or remain at home longer before being brought to the hospital or other care facility, which may reduce stress and discomfort to the patient and may also be more cost-effective.

In FIG. 1, telemedicine system 100 includes a remote visualization system 102, which includes components at a first location 104, and a medical monitoring system 106, which includes components at a second location 108 remote from first location 104. First location 104 is the location at which the subject 110 (e.g., a patient) receives monitoring, treatment, or consultation, and may be, for example, the subject's home, or a satellite medical office or clinic. Second location 108 is the location from which medical consultation and/or supervision is provided. For example, in FIG. 1, subject 110 and a caregiver 112 (e.g., a home health nurse) are depicted at the first location 104, while a medical care provider 114 (e.g. a physician) is depicted at second location 108. Medical care provider 114 may be a physician (e.g., a general practitioner or specialist), physician's assistant, nurse practioner, nurse, dentist, or any of type medical care provider, without limitation. Caregiver 112 may be a home health nurse, a nursing assistant, a family member, or any other party who assists in care of subject 110. The presence of a caregiver 112 is optional and in many cases, subject 110 may receive medical care and interact with medical care provider 114 through the use of telemedicine system 100 independently, without assistance from caregiver 112. Either subject 110 or caregiver 112 may be a user of remote visualization system 102.

Remote visualization system 102 includes an audio input device 120 at first location 104 and imaging system 122 at the first location 102 adapted to acquire an image 124 of subject 110, image 124 containing information indicative of a health status of the subject. A health status may include, but is not limited to, one or more of a medical condition, a medical state, a healthy state, a diseased state, an injured state, a mental health state, a physical health state, a normal state, an unremarkable state, and a symptomatic state, for example. A health status may include a status that is unremarkable (e.g. normal and healthy, and/or normal for the patient), or that is normal but noteworthy (e.g., the subject is pregnant). A health status may include disease state, disease progression, disease regression, wound healing or lack thereof, signs of infection, or increase or decrease in symptoms, for example. Information indicative of a health status may include information indicative of a symptom, a vital sign, the subject's mood, and various other information as described elsewhere herein or as known to those skilled in the relevant arts. Remote visualization system 102 also includes video output device 126 at first location 104; and audio output device 128 at first location 104. Remote visualization system 102 includes controllable lighting system 130 including at least one light source 132 (here, four LED sources are shown which are components of light source 132, which is a compound light ource) adapted to illuminate at least a portion of subject 110 during acquisition of image 124 of the subject, image 124 containing information indicative of the health status of the subject and having at least one controllable parameter that influences at least one of the amount or type of information indicative of the health status of the subject in an acquired image 124 of the subject. The intensity of light provided by light sources 132 of controllable lighting system 130 is controlled by the amount of current driving each light source, which in turn is controlled by first electrical control circuitry 134.

Remote visualization system 102 includes first electrical control circuitry 134 at first location 104 operatively connected to and configured to control operation of the audio input device 120, imaging system 122, video output device 126, audio output device 128, and controllable lighting system 130. In the embodiment of FIG. 1, electrical control circuitry 134 includes an appropriately configured computer. Imaging system 122 includes a camera configured to be built into, attached to, or used in combination with a computer monitor (e.g. for use as a “webcam”). Such cameras are commercially available and well known to those skilled in the art. In the embodiment of FIG. 1, audio input device 120 is a microphone adapted to plug into an audio jack of a computer, video output device 126 is a computer monitor, and audio output device 128 is a speaker (which may be built into the monitor, as depicted here, or packaged as a separate unit for use with the computer).

In addition, remote visualization system 102 includes communication circuitry 136 at first location 104 configured to provide communication between first electrical control circuitry 134 and second electrical control circuitry 140 at a second location 108 remote from the first location 104 via communication link 142. A representation of image 124 is transmitted from first location 104 to second location 106 as image data signal 144. In order to provide remote communication between medical care provider 114 and subject 110 and/or caregiver 112, audiovisual data signals 150 are transmitted via communication link 142, from audio input device 120 and imaging system 122 to communication circuitry in second electrical circuitry 140, where they may be presented to medical care provider 114 via video output device 152 and audio output device 154. Similarly, audiovisual data signals 156 from imaging system 158 and audio input device 160 at second location 108 are transmitted to first location 104 via communication link 142, where they may be presented to patient 110 and/or caregiver 112 via video output device 126 and audio output device 128. In an aspect, audio and visual information contained in audiovisual data signals 150 and 156 is used for teleconferencing purposes, to provide for communication between patient 110 and/or caregiver 112 and medical care provider 114. In addition, in some aspects visual information contained in audiovisual data signal 150 may be subjected to additional processing and/or analysis for the purpose of extracting medically useful information from images contained in audiovisual data signal 150.

In the example shown in FIG. 1, image 124 is acquired by imaging system 158 at a first lighting condition. Image data signal 144 may be part of audiovisual data 150, or it may be a separate data signal, e.g. a data signal obtained under different conditions (under different lighting conditions, at a different magnification, at a different frame rate, etc.) having a different data format (e.g. different resolution) and/or stored or processed separately from audiovisual data 150. Image 124 is acquired for the purpose of providing information indicative of the health status of subject 110 to medical care provider 114. An image of the subject may provide medically useful information regarding many aspects of a subject's health and condition.

In an aspect, the amount or type of information in image 124 is influenced by one or more parameters of the lighting delivered by controllable lighting system 130. Accordingly, analysis of the information content of image 124 may indicate the need to adjust the lighting provided by controllable lighting system 130. For example, in order to more clearly visualize certain features of subject 110 in the acquired image, it may be determined that a higher level of lighting is needed. Alternatively, or in addition, it may be determined that a different wavelength of light should be used to provide better visualization of certain features of subject 110. In the system depicted in FIG. 1, controllable lighting system 130 includes multiple light sources 132, capable of delivering light of several different wavelengths. The intensity of light delivered by each light source can be varied under the control of electrical control circuitry 134.

Electrical control circuitry 134 is configured to control the controllable parameter of the controllable lighting system 130 responsive to receipt of a lighting control signal 146 from second electrical control circuitry 140 at second location 108. As will be discussed in greater detail elsewhere herein, electrical control circuitry 140 generates lighting control signal 146 by analyzing image data signal 144 to determine an adjustment to controllable lighting system 130 to influence the amount and type of information in images acquired by imaging system 122. Following adjustment of controllable lighting system 130 to modify the lighting condition, a subsequent image 148 may be acquired at a second lighting condition and information indicative of the health status of the subject obtained from the image. It will be appreciated that if the desired information content is not obtained following the initial adjustment of controllable lighting system 130, further image analysis followed by additional adjustment to lighting system 130 may be performed.

Remote visualization system 102 may include and/or be used in combination with an article of medical equipment, e.g. blood pressure cuff 162 and blood pressure measuring system 164, which may communicate with electrical control circuitry 134. In an aspect, diagnostic data 170 transmitted to control circuitry 140 at second location 108 via communication link 142 includes blood pressure data obtained from blood pressure measuring system 164. In an aspect, control signal 172 sent from second electrical control circuitry 140 via communication link 142 includes one or more control signals for controlling blood pressure measuring system 164. In an aspect, blood pressure measuring system 164 is controlled based on information contained in image data signal 144. For example, if analysis of image data signal 144 indicated that patient 110 had become paler than usual, blood pressure measuring system 164 could be controlled to take a blood pressure measurement in order to detect whether the patient's paleness corresponded to a drop in blood pressure.

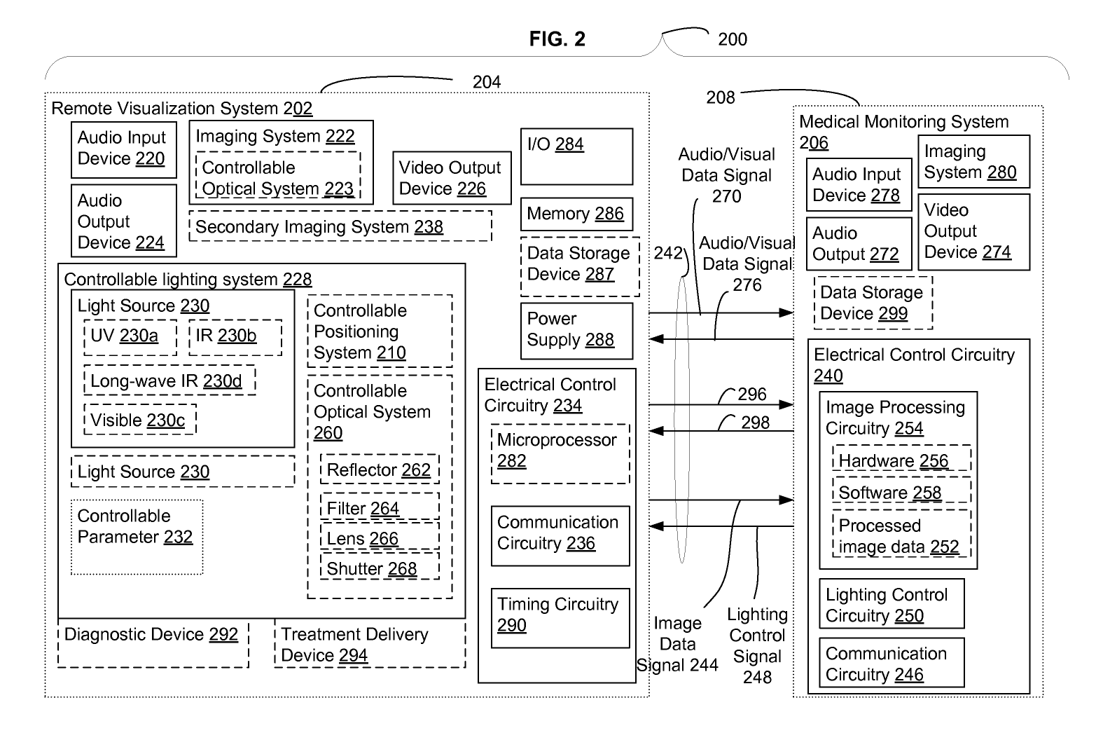

FIG. 2 is a block diagram of generalized telemedicine system 200 of which the system depicted in FIG. 1 is an example. Telemedicine system 200 includes remote visualization system 202 at a first location 204 (the patient location) and medical monitoring system 206 at a second location 208 (the medical monitoring location), which is remote from first location 204. Remote visualization system 202 includes audio input device 220, imaging system 222, audio output device 224, video output device 226, and controllable lighting system 228, which includes at least one light source 230 and has at least one controllable parameter 232 that influences at least one of the amount or type of information indicative of the health status of the subject in an acquired image of the subject. In this and other figures, dashed lines indicate optional or alternative components.

Remote visualization system 202 also includes electrical control circuitry 234, which is operatively connected to and configured to control operation of the audio input device 220, imaging system 222, audio output device 224, video output device 226, and controllable lighting system 228. Remote visualization system also includes communication circuitry 236 at first location 204, which is configured to provide communication between the electrical control circuitry 234 and electrical control circuitry 240 at location 208, via communication link 242.

A representation of an image acquired by imaging system 222 is transmitted from first location 204 to second location 208 as image data signal 244. Audiovisual data signals 270 are transmitted via communication link 242, from audio input device 220 and imaging system 222 to communication circuitry 246 in second electrical circuitry 240, where they may be presented to a medical care provider via video output device 274 and audio output device 272. Similarly audiovisual data signals 276 from imaging system 280 and audio input device 278 at second location 208 are transmitted to first location 204 via communication link 242, where they may be presented to a patient and/or caregiver via video output device 226 and audio output device 224. Two-way audio-visual communication between caregiver and patient/caregiver may be used for video conferencing purposes associated with telemedicine, e.g., for remote consultation, asking and answering of questions, offering of medical advice and instructions, etc.

In a general sense, those skilled in the art will recognize that the various embodiments described herein can be implemented, individually and/or collectively, by various types of electrical circuitry having a wide range of electrical components such as hardware, software, firmware, and/or virtually any combination thereof Electrical circuitry (including electrical control circuitry 234 and electrical control circuitry 240 depicted in FIG. 2, for example) includes electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor (e.g. microprocessor 282) configured by a computer program which at least partially carries out processes and/or devices described herein), electrical circuitry forming a memory device (for example, memory 286, which may include various types of memory (e.g., random access, flash, read only, etc.)), electrical circuitry forming a communications device (e.g. communication circuitry 236 or 246) (e.g., a modem, communications switch, optical-electrical equipment, etc.), and/or any non-electrical analog thereto, such as optical or other analogs (e.g., graphene based circuitry). In a general sense, those skilled in the art will recognize that the various aspects described herein which can be implemented, individually and/or collectively, by a wide range of hardware, software, firmware, and/or any combination thereof can be viewed as being composed of various types of “electrical circuitry.”

Those skilled in the art will recognize that at least a portion of the devices and/or processes described herein can be integrated into a data processing system. Those having skill in the art will recognize that a data processing system generally includes one or more of a system unit housing, a video display, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

In an aspect, remote visualization system 202 includes at least one data storage device 287, which may include and/or be the same as memory 286, or which may be a separate data storage device used in addition to memory 286, to store data at first location 204.

Audio input device 220 and audio input device 278 may include a microphone, for example. Various microphones suitable for transducing voice signals are known to those having skill in the art, and may be suitable for telemedicine purposes. Audio input devices 220 and 278 may be configured as separately packaged devices configured to communicate with electrical control circuitry (234 or 240, respectively) via a wired connection (via a plug and jack or USB, for example) or wireless connection. Alternatively, audio input devices 220 and 278 may be built in or packaged with other system components. Electrical control circuitry 234 may further include additional input and output devices, e.g. I/O 284.

In an aspect, a single imaging system 222 is used. In an aspect two or more imaging systems may be used, e.g. secondary imaging system 238 shown in FIG. 2. Imaging system 222 and secondary imaging system 238 may be the same or different types of imaging systems. In an aspect one imaging system may be used to detect video images for use in audio/visual communication between patient and medical care provider, and the other imaging system may be used to detect still or moving images of all or a portion of the subject for medical diagnostic purposes. For example, a conventional commercially available video camera suitable for video conferencing can be used for audio/visual communication between patient and medical care provider. In an aspect, the camera for audio/visual communication between patient and medical care provider may also provide medically useful information. In an aspect, two or more cameras may be used to provide views of the subject from two or more different angles or positions. In an aspect, a specialized camera may be used to obtain images for medical diagnostic purposes. For example, a specialized camera may produce images at a particular wavelength or range of wavelengths of light, have a higher spatial resolution or higher frame rate, or have other characteristics that permit it to obtain medically useful information, for example as described in U.S. Patent Publication 20120307056 dated Dec. 6, 2012 to Zuzak et al., and U.S. Patent Publication 201230128223 dated May 23, 2013 to Wood, each of which is incorporated herein by reference. The camera may function as a fundus camera or dermatoscope, for example. For example, a specialized camera may be sensitive to particular wavelengths of light, due either to the spectral sensitivity of light detecting elements of the camera or through the use of filters to limit the wavelengths of light sensed. A specialized camera may include wavelength or polarization dependent filters to select the wavelength, wavelength band, and/or polarization of light to be imaged, or a lens system to provide magnification. One or both of imaging system 220 and secondary imaging system 238 may include one or more photocell, charge-coupled device, scanner, 3D scanner, 3D imager, camera, single pixel camera, a visual camera, IR camera, a stereoscopic camera, a digital camera, a video camera, and a high speed video camera, for example. One or more digital images of the skin surface of the subject for use in generating a digital three-dimensional representation of the skin surface can be acquired from one or more of a digital camera or scanning device. For example, two video cameras, slightly apart, can be used to image the same portion of skin surface of the individual in a process termed stereophotogrammetry. For example, a single camera can be used to take multiple images under different lighting conditions or from different positions. In an aspect, the topography of the skin surface of an individual can be acquired in a point-cloud format using a three-dimensional sensing system consisting of two or more digital cameras and one or more projectors connected to a personal computer. The camera position and shutter can be adjusted to the body region, which is exposed to structured light, allowing for optical representation of the surface by a cloud of up to 300,000 points in three-dimensional coordinates (see, e.g., Feng et al., Br. J. Oral Maxillofac. Surg. (2010) 48:105-109, which is incorporated herein by reference). In some embodiments, the combination of stereophotogrammetry and 3D laser scanner techniques can be combined to generate a three-dimensional model of the skin surface of an individual (see, e.g., Majid, et al. International Archives of the Photogrammetry, Remote Sensing and Spatial Information Science. Vol.)(XXVII. Part B5. (2008) 805-811; Markiewicz & Bell, Facial Plast. Surg. Clin. N. Am. (2011) 19:655-682; van Heerbeek et al., Rhinology (2009) 47:121-125, which are incorporated herein by reference). Scanners for scanning head, face and/or whole body are commercially available (from, e.g., Cyberware, Monterery Calif.; Accurex Measurement Inc., Swathmore, Pa.; 3dMD Atlanta, Ga.; Konica/Minolta, Ramsey, N.J.). In an aspect, remote visualization system 202 may function, for example, as a fundus camera or dermatoscope. Remote visualization system 202 may include one, two, or more imaging systems, which may be all of the same type or of different types, including but not limited to the types of imaging systems listed above. Imaging system 222 may be a controllable imaging system. In an aspect, imaging system 222 may include a controllable optical system 223, which may include one or more controllable components such as reflectors, filters, lenses, or shutters, which may be used to control various aspects of the image detected by imaging system 222. In some aspects, the imaging system may include controllable positioned components for adjusting the position of the imaging system. Filtration, pan, tilt, or zoom of the imaging system may be controlled by adjustment of these and/or other controllable components, for example. Controllable optical system 223 may be controlled by electrical control circuitry 234. In an aspect, controllable optical system 223 may be controlled in response to control signals from electrical control circuitry 240 in medical monitoring system 206.

Medically useful information regarding a subject's health and/or health status may include overall or localized color or texture of the subject's skin, puffiness or swelling of the subject's features, color, presence, size, or shape of features of the subject's skin (e.g., moles, rashes, blemishes, bruises, wounds, sores, scars). Such medically useful information may be indicative of the health status of the subject. See, for example, U.S. Pat. No. 7,894,651 issued Feb. 22, 2011 to Gutkowicsz-Krusin et al.; U.S. Patent Publication 2012/0157800 dated Jun. 21, 2012 to Tschen; U.S. Patent Publication 2012/0268462 dated Oct. 25, 2012 to Sota et al.; U.S. Patent Publication 2012/0041275 published Feb. 16, 2012 to Sota et al.; U.S. Pat. No. 7,289,211 issued Oct. 30, 2007 to Walsh, Jr. et al.; U.S. Patent Publication 2012/0224753 published Sep. 6, 2012 to Bogdan; U.S. Patent Publication 2011/0064287 published Mar. 17, 2011 to Bogdan: U.S. Pat. No. 8,208,698 issued Jun. 26, 2012 to Bogdan; U.S. Patent Publication 2009/0154781 published Jun. 18, 2009 to Bogdan; and U.S. Patent Publication 2008/0275315 published Nov. 6, 2008 to Oka et al., each of which is incorporated herein by reference. Image 24 may be a portion of a video, such that analysis of image 24 within the context of other images from the video, e.g. to detect motion, may provide information relating to respiration, pulse, tremors, etc. that may inform the medical caregiver 114 regarding the condition of subject 110. See, e.g. U.S. Patent Publication 2009/0306487 published Dec. 10, 2009 to Crowe et al. Medically useful information may be obtained from images of a subject's eyes. For example, retinal scans may be used in the detection of glaucoma, diabetes, or drug use. See, for example the camera systems described in U.S. Patent Publication 20130083185 dated Apr. 4, 2013 to Coelmen III, and U.S. Patent Publication 2012-0101371 dated Apr. 26, 2012 to Verdooner, each of which is incorporated herein by reference.

In an aspect, at least one of video output device 226 and video output device 274 includes at least one video display device. Video display devices include, but are not limited to, computer monitors, displays built into dedicated medical monitoring devices, displays built into smart phones or table computers, for example. A variety of displays having size, resolution, frame rate, suitable for video conferencing applications are known to those skilled in the relevant art. In an aspect, at least one of audio output device 224 and audio output device 272 includes at least one speaker, which as discussed in connection with FIG. 1 may be a separately packaged unit adapted to plug into a computer system or other system, or may be built into another computer system component, a dedicated medical monitoring system, a smart phone, a table computer, etc.

Electrical control circuitry 234 in remote visualization system 202 is configured to control controllable parameter 232 of controllable lighting system 228 responsive to receipt of a lighting control signal 248 from the electrical control circuitry 240 in medical monitoring system 206. Lighting control signal 248 may specify adjustment to one or more of a variety of parameters, including but not limited to intensity, aiming, light pulse, divergence or convergence, spectral content, or polarization of light from the at least one controllable light source, adjustment to a position of the at least one controllable light source, for example. Controllable parameter 232 may be, for example, a light intensity, wavelength, wavelength band, polarization orientation or range of orientations. In some aspects, these parameters may be controlled by controlling the operation of the light source directly. In some aspects, the parameter may be controlled by controlling which of several light sources produce light, or by controlling filters, reflectors, lenses, shutters, or other optical components to modify light produced by one or more light sources.

Medical monitoring system 206 includes electrical control circuitry 240 at second location 208 adapted to communicate with first electrical control circuitry 234 at first location 204 via communication circuitry 246 and 236.

Communication circuitry 236 in remote visualization system 202 and communication circuitry 246 in medical monitoring system are configured to provide a communication link 242 between the two locations. Communication link 242 may be any of various types of communication links suitable for providing communication between two remote locations. Communication between locations remote from each other may take place over telecommunications networks, for example public or private Wide Area Network (WAN). In general, communication between remote locations is not considered to be suitably handled by technologies geared towards physically localized networks, e.g. Local Area Network (LAN) technologies operation at Layer ½ (such as the forms of Ethernet or WiFi). However, it will be appreciated that portions (but not the entirety) of communication networks used in remote communications may include technologies suitable for use in physically localized network, such as Ethernet or WiFi. In an aspect, system components are considered “remote” from each other if they are not within the same room, building, or campus. In an aspect, a remote system may include components separated by a few miles or more. Conversely, system components may be considered “local” to each other if they are located within the same room, building, or campus.

In an aspect communication circuitry 236 is configured to provide communication with the second electrical control circuitry at the second location via a wireless communication link. In an aspect, the communication circuitry 236 is configured to provide communication with the second electrical control circuitry 240 at the second location 208 via a cellular communication link. In an aspect, the communication circuitry 236 is configured to provide communication with the second electrical control circuitry 240 at the second location 208 via a WiFi communication link. In various aspects, the wireless communication link includes at least one of a radiowave, wireless network, cellular network, satellite, WiFi, Wide Area Network, Local Area Network, or Body Area Network communication link.

In an aspect, the communication circuitry 236 is configured to provide wireless communication between at least two system components at first location 204. In an aspect, communication circuitry 236 is configured to provide wired communication between at least two system components at the first location 204. System components connected via wired or wireless connections may include, but are not limited to, audio input device 220, imaging system 222, audio output device 224, video output device 226, controllable lighting system 228, electrical control circuitry 234, diagnostic device 292, and treatment delivery device 294, for example.

Lighting control signal 248 is generated by lighting parameter control circuitry 250 in response to processed image data 252 from image processing circuitry 254. For example, second electrical control circuitry at the second location may include image processing circuitry 254 adapted to process the acquired image to determine at least one feature of the acquired image, lighting parameter control circuitry 250 adapted to determine an adjustment of the at least one controllable parameter of the controllable lighting system at the first location based on the at least one feature of the acquired image, and communication circuitry for controlling transmission of the lighting control signal for controlling the adjustment of the at least one controllable parameter from the second location to the first location via the communication circuitry.

In an aspect, electrical control circuitry 240 at second location 208 includes image processing circuitry 254, which may include hardware 256 and/or software 258. Image processing circuitry 254 receives image data signal 244 and performs image processing to determine (e.g. detect and/or quantify) features of all or portions of the image. Features may include, but are not limited to, brightness, spatial frequency composition, and contrast. Image features may be determined at one or more wavelength or wavelength range and/or one or more polarization or range of polarizations. Image features may include, but are not limited to, edges, corners, ridges, and other shapes within the image. Images features may be indicative of texture, color, granularity, among others. Various types of features may be determined, without limitation, using methods known to those skilled in the art of image processing and analysis. See, for example, U.S. Pat. No. 7,894,651 issued Feb. 22, 2011 to Gutkowicsz-Krusin et al. describing characterization of lesions based on measurements of lesion asymmetry, border irregularity, color variegation, and diameter; U.S. Pat. No. 7,289,211 issued Oct. 30, 2007 to Walsh, Jr. et al. describing distinguishing tissue types based on Stokes polarimetry; and U.S. Patent Publication 2012/0224753 published Sep. 6, 2012 to Bogdan, describing characterization of lesion texture based on wavelet analysis; all of which are incorporated herein by reference.

Image processing performed by image processing circuitry 254 may include filtering, noise reduction, feature extraction, pattern recognition, projection, multi-scale signal analysis, pixelation, scaling, classification, component analysis, or Hidden Markov models, for example.

In various aspects, image processing circuitry 254 is adapted to process the acquired image to determine at least one of brightness, contrast, or spatial frequency content of at least a portion of the acquired image, at one or more wavelength or wavelength band, and/or at one or more polarization or range of polarizations.

In various aspects, the image processing circuitry 254 is adapted to process the acquired image to detect at least one of a shape, a line, a corner, and an edge within at least a portion of the acquired image. Controllable lighting system 228 can include at least one light source 230 (two light sources 230 are depicted in FIG. 2, but other numbers of light sources may be used in various embodiments). Light source 230 may include, but is not limited to, ultraviolet light source 230a, infrared light source 230b, visible light source 230c, or long-wave infrared 230d light source, for example. Light sources capable of generating light in these wavelengths are well known to those having ordinary skill in the art, and may include, for example LEDs, lasers, laser diodes, incandescent light sources, or fluorescent light sources. The choice of light source will depend on the type of medical information that is to be obtained from the image. For example, a broad spectrum or white light source used in combination with an RGB camera can be used in detection of malignant melanoma (See U.S. Pat. No. 7,894,651 issued Feb. 22, 2011 to Gutkowicsz-Krusin et al., which is incorporated herein by reference), Infrared or blue spectrum light may also be used in the detection of melanoma based on lesion texture (see U.S. Pat. No. 8,208,698 issued Jun. 26, 2012 to Bogdan and U.S. Pat. No. 7,289,211 issued Oct. 30, 2007 to Walsh, Jr. et al. both of which are incorporated herein by reference, and the latter of which describes the use of various wavelengths, including white light, 940 nm, Near IR, etc. in detection of tissue type and structure). Green light (e.g. at 510 nm) may enhance detection of blood vessels (see U.S. Patent Publication 2009/0306487 published Dec. 10, 2009 to Crowe et al., which is incorporated herein by reference). In an aspect, light source 230 may have one or more of a controllable intensity, controllable spectral content, or controllable polarization. Intensity of emitted light is typically controlled by amplitude of the electrical driving signal. Intensity, spectral content, and/or polarization can be controlled with the use of filters, or by controlling operation or selection of light source.

In an aspect, controllable lighting system 228 includes a controllable optical system 260, which may include one or more optical components, e.g., reflector 262, filter 264, lens 266, or shutter 268. Filter 264 may filter out light of one or multiple wavelengths or wavebands and/or filter light of a particular polarization or range of polarities. In an aspect, first electrical control circuitry 234 is configured to control the at least one filter to control at least one polarization (e.g. orientation of polarization) of light delivered by the controllable lighting system.

In an aspect, controllable lighting system 228 is adapted to generate a light pulse having a pulse duration. In connection therewith, in an aspect electrical control circuitry 234 is configured to control generation of the light pulse by controllable lighting system 228. Controlling generation of the light pulse may include controlling the pulse duration of the light pulse and/or amplitude (intensity) of the light pulse, as well as controlling time of delivery of the light pulse. Timing of delivery of a light pulse can be performed under the control of electrical control circuitry 234 through the use of timing circuitry 290 (which may include a clock, timer, or counter device), using methods known to those having skill in the art.

Electrical control circuitry may be configured to coordinate detection of an image by the imaging system with generation of a light pulse by the controllable lighting system 228. In an aspect, an image may be detected by a compressive imaging technique in which a short, brief pulse of light is delivered to one or more portions of the region to be imaged, and an image detected at the time that the portions of the region to be imaged are illuminated by the pulse of light (i.e., simultaneously or substantially simultaneously with the generation of the light pulse). Compressive imaging may be used to reduce energy consumption, light exposure to imaged area, and memory used for image data storage. Compressive imaging may be performed with a single pixel camera or multi-pixel camera. See, e.g. U.S. Pat. No. 8,199,244 issued Jun. 12, 2012 to Baraniuk et al. and U.S. Pat. No. 8,125,549 issued Feb. 28, 2012 to Deket, both of which are incorporated herein by reference.

In an aspect, controllable lighting system 228 includes a controllable positioning system 210. In an aspect, controllable positioning system 210 is adapted to adjust the aiming of light produced by the at least one light source. Controllable positioning system 210 may include a mechanical linkage (e.g. jointed or telescoping arm) to which one or more light source 230 is attached, or light source 230 may be mounted on a movable mounting that moves with respect to a support (e.g by rotation or translation) to adjust the position of light source 230. In an aspect, controllable positioning system 210 is adapted to scan a beam of light produced by the at least one light source across at least a portion of the subject. This can be done, for example, with a controllable mirror or reflector. Controllable positioning system may control both position, orientation, and aiming of at least one light source 230.

As shown in FIG. 2, in an aspect, remote visualization system 204 includes or can be used in combination with at least one diagnostic device 292 adapted for detecting diagnostic data indicative of a health status of a subject. Diagnostic device 292 may include, for example one or more of a blood pressure cuff, a thermometer, a stethoscope, an electrocardiogram (ECG) monitor, an electroencephalogram (EEG) monitor, a bioelectromagnetic sensor for sensing one or more bioelectric or biomagnetic signals (including but not limited to electroencephalogram, electrocardiogram, electromyogram, electrooculogram, magnetic counterparts thereof), an ultrasound probe, a chemical sensor (e.g. for measuring chemicals or gases in bodily fluids in fluid samples taken from the body or within the body, including but not limited to blood, plasma, serum, saliva, urine, mucus, tears, semen, and vaginal secretions), a gas sensor (for measuring blood gases, expired gases, flatus, etc.) a touch probe, or a bed mat sensor. In an aspect, first electrical control circuitry 234 is configured to receive information from at least one diagnostic device 292 at first location 204.

In an aspect, system 204 includes or is used in combination with at least one treatment delivery device 294, which may be, for example, a substance delivery device, e.g. controllable medication dispensing device configured to dispense at least one formulated medication in response to a control signal from the first electrical control circuitry 234 (which may be, for example, a pill dispenser of the type described in U.S. Pat. No. 8,452,446 issued May 28, 2013 to Madras et al., which is incorporated herein by reference), or other device configured to dispense pills, capsules, powders, liquids, inhalants, and other oral medications or inhalable medications. A medication dispenser may also deliver formulated medications for topical delivery, such as creams, ointments, eye drops, etc. In an aspect, system 204 includes or is used in combination with at least one transdermal substance delivery device, including for example, one or more of an injection device, a needle-based injection device configured to deliver an injectable substance in response to a control signal from the first electrical control circuitry 234 (e.g. as described in U.S. Pat. No. 6,056,716 issued May 2, 2000 to D'Antonio et al. and U.S. Pat. No. 8,544,645 issued Oct. 1, 2013 to Edwards et al., both of which are incorporated herein by reference), a needleless injection device, an air gun, a jet injector, microneedles, a patch, or an infusion system configured to deliver an infusible substance in response to a control signal from the electrical control circuitry 234 (e.g., of the type described in U.S. Pat. No. 8,348,885 issued Jan. 8, 2013 to Moberg et al., which is incorporated herein by reference). In other aspects, treatment delivery device 294 may be configured to deliver other types of treatments to the subject, for example, delivery of various forms of energy (light, electrical, magnetic, electromagnetic, acoustic, ultrasonic, thermal), pressure, vibration, cooling (i.e., removal of energy), to produce various therapeutic effects in the subject. Treatment delivery device may include one or more electrodes, light sources, electromagnetic field sources, piezoelectric devices, magnets, electromagnets, heating elements, for example, In an aspect, first electrical control circuitry 234 is configured to send a control signal to at least one treatment delivery device 294 at the first location. In an aspect, combination of one or more diagnostic devices 292 and/or treatment delivery devices 294 with remote visualization system 202 allows more complete medical care to be provided to the subject. In an aspect, data signals 296 transmitted from location 204 to medical monitoring system 208 via communication channel 242 include diagnostic data and/or status signals from diagnostic device 292 and substance delivery device 294. In addition, control signal 298 from electrical control circuitry 240 in medical monitoring system 206 include signals for controlling diagnostic device 292 and treatment delivery device 294. In an aspect, electrical control circuitry 234 is configured to control receipt of diagnostic data indicative of a health status of the subject from the at least one diagnostic device 292 at first location 204. In an aspect, electrical control circuitry 234 is configured to control transmission of a signal indicative of the diagnostic data to second location 208.

As noted in connection with FIG. 1, in some aspects, control signal 298 is determined by electrical control circuitry 240 based fully or in part upon analysis of image data signal 244, e.g. such that one or more diagnostic device 292 and/or substance delivery device 294 are controlled responsive to the patient's health status as determined from medically useful information in image data signal 244. For example, image data signal 244 may be analyzed to determine whether a subject has taken a dispensed dose of medication. It will be appreciated that various device control signals, data signals, instructions, status signal, and the like may be transmitted between medical monitoring system 206 and remote visualization system 202 other than those explicitly recited herein.

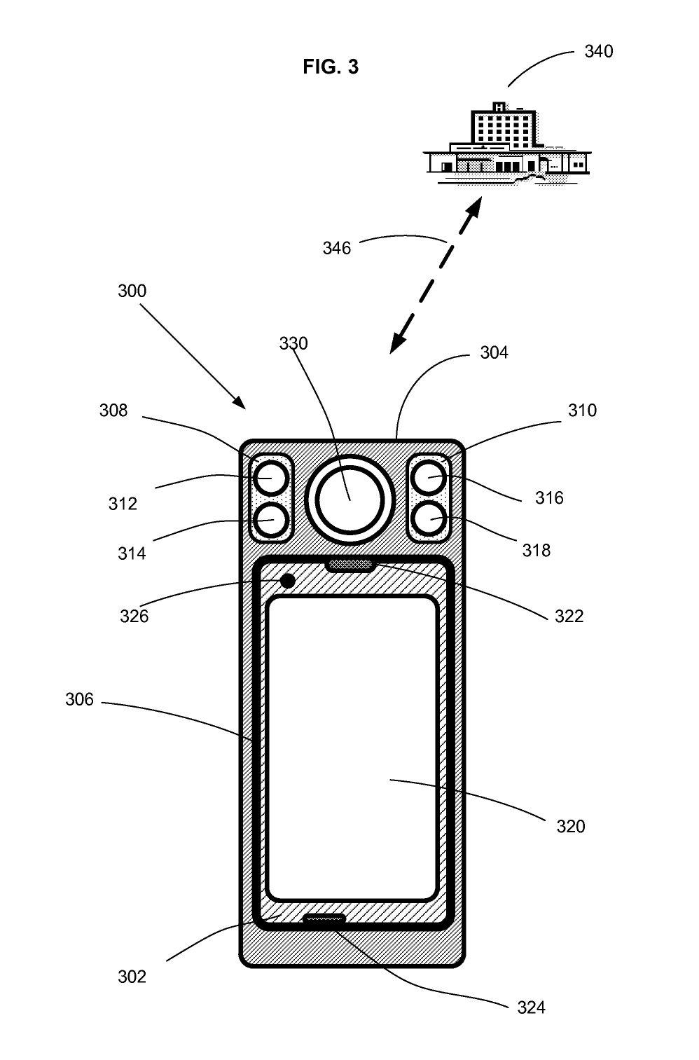

FIG. 3 depicts an embodiment in which at least a portion of a remote visualization system is configured as a hand-held unit. In the example of FIG. 3, remote visualization system 300 includes a smart phone 302 mounted in housing 304, which includes a recess 306 adapted to receive smart phone 302. Recess 306 is shaped to receive smart phone 302 to physically attach housing 304 to smart phone 302 so that smart phone 302 and housing 304 function as a hand-held unit. In an aspect, recess 306 also includes data and power connections for sending or receiving data and power signals between housing 304 and smart phone 302. Housing 304 also includes controllable compound light sources 308 and 310. Compound light source 308 includes two individual component light sources, 312 and 314, and compound light source includes two individual component light sources, 316 and 318. Light sources 308 and 310 together form a controllable lighting system. Housing 304 also includes camera 330, which may have the same or different properties than smartphone camera 326. Housing 304 further includes electrical circuitry adapted to control operation of light sources 308 and 310 and camera 330, in response to instructions received from smart phone 302 via data and power connections. Housing 304 may include a lens configured to be used in combination with the camera 326 built into smart phone 302, as described generally in Design Patent D669587, issued Oct. 23, 2012 to Mayer, and U.S. Patent Publication 2008/0275315 Published Nov. 6, 2008 to Oka et al., each of which is incorporated herein by reference.

Handheld unit 300, which functions as the remote visualization portion of a telemedicine system, thus includes video display 320, speaker 322, microphone 324, which are components of smartphone 302, as well as controllable compound light sources 308 and 310. One or both of camera 326 in smartphone 302 and camera 330 in housing 304 function as imaging systems for obtaining images containing medically useful information. As can be seen, controllable compound light source 308 and controllable compound light source 310 differ with regard to position. Furthermore, light sources 312 and 316 are broad band light sources adapted to produce light containing a range of wavelengths, while light sources 314 and 318 are near infrared light sources. By selecting the appropriate light source, the wavelength of light delivered to the subject can be controlled. It will be appreciated that a patient or caregiver can use the smartphone in video conferencing mode to communicate with a medical care provider at a remote location (hospital 340). If it is desired to image a portion of the patient's body other than the portion visible while the patient is speaking to the medical care provider via the smart phone video conference, the patient or caregiver can temporarily halt or pause the video conference and aim the handheld unit toward the portion of the body to be imaged in order to obtain the desired image. Lighting provided by controllable compound light sources 308 and 310 can be controlled at least in part by electrical control circuitry located at hospital 340, to provide lighting conditions to obtain the desired medical information from the acquired image, as described elsewhere herein. Communication between handheld unit 300 and a medical monitoring system at hospital 340 can be carried out over a cellular network 346 (it will be appreciated that cellular network 346 may include one or more base stations and/or transceivers between handheld unit 300 and hospital 346, as are well known to those of ordinary skill in the art, although these are not depicted in FIG. 3). As illustrated in FIG. 3, in an aspect, a controllable lighting system can include at least one compound light source, the compound light source including at least two individual light sources. In various aspects, the at least two individual light sources forming the compound light source differ from each with regard to at least one of position, waveband and polarization of light produced. Individual light sources forming a compound light source may be activated separately or simultaneously to produce light. In an aspect, the characteristics of light produced by a compound light source may be varied by selectively activating the individual light source to select a desired combination of wavelength, polarization, intensity, and so forth.

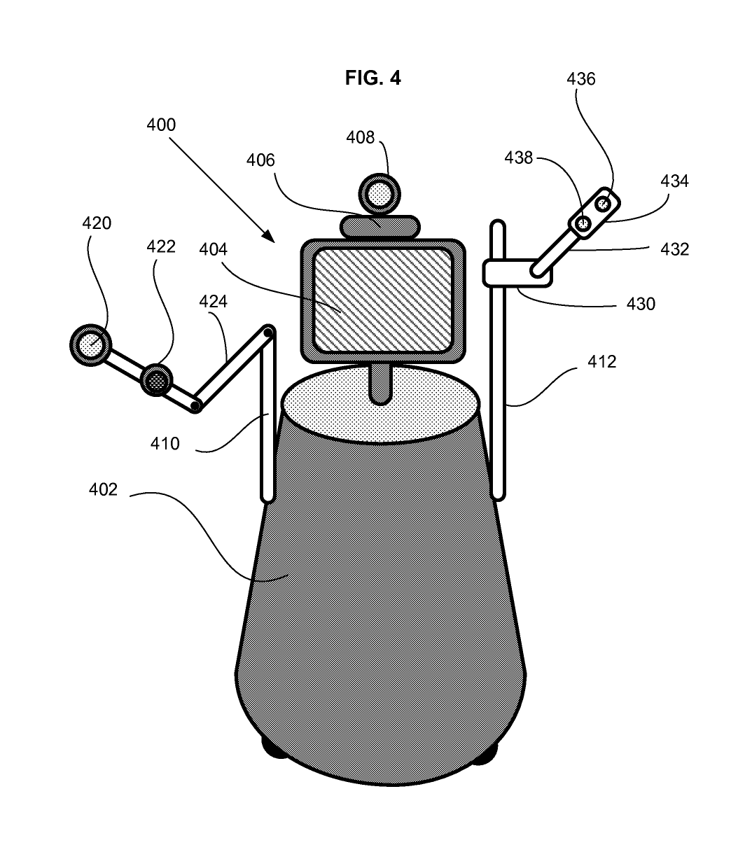

FIG. 4 illustrates an example in which at least a portion of the remote visualization system is configured as a mobile robot 400. Mobile robot 400 includes a mobile base 402, a video display 404, speaker 406 and camera 408. In an aspect, mobile robot is of the type described, for example in U.S. Patent Publication 2012/0197439 published Aug. 2, 2012 to Wang et al., which is incorporated herein by reference. In an aspect, mobile robot 400 can be anthropomorphized in one or more features. Mobile robot further includes mounting structures 410 and 412, which can be used to support and position selected system components. Mounting structures 410 and 412 are depicted as mounting poles, but in other aspects mounting structures can be any type of structure to which system components can be secured and supported or suspended in a desired position, including for example, poles, arms, mechanical linkages, goosenecks, cables, chains, etc.) In the example depicted in

FIG. 4, mounting structure 410 includes camera 420 and microphone 422 mounted on linkage 424, which provides for positioning camera 420 and microphone 422 close to a patient who may be, for example, lying in a bed or sitting in a chair. Mounting 430 is attached to mounting structure 412. A further linkage arm 432 is attached to mounting 430. Controllable lighting system 434, which includes two light sources 436 and 438, is attached to linkage arm 432. Mounting structures 410 and 412 and associated mechanical linkages 424 and 432 and mounting 430 may be operated under the control of one or more of control circuitry in mobile robot 400 or control circuitry at a remote location. Mounting structure may be manually extendable, or extendable under control of the electrical control circuitry.

Microphone 422, a video display 404, speaker 406 and one or both of camera 408 and 420 provide the audio and video inputs and outputs necessary for audiovisual communication between a patient at the location of mobile robot 400 and a remote medical care provider. Either camera 408 or camera 420 may be used as an imaging system for obtaining an image containing medically useful information. Variations of the configuration depicted in FIG. 4 are possible. In an aspect, a mounting structure may be configured to support at least one of the controllable lighting system 434, the imaging system (including one or both of camera 408 and 420), the video output device (video display 404), audio input device (microphone 422), and the audio output device (speaker 406).

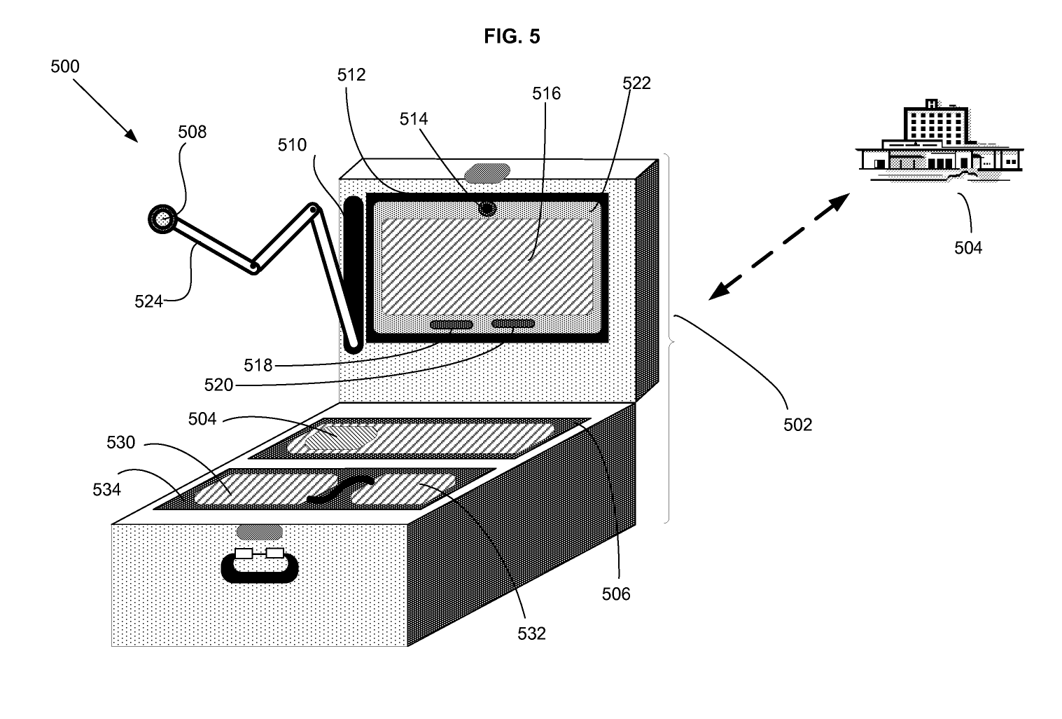

FIG. 5 depicts an embodiment in which at least a portion of the remote visualization system is configured in a container 502. For example, remote visualization system 500 including container 502 is suitable for being provided to a patient upon release from a hospital 504, for transport to the patient's home and use by the patient with remote monitoring by a medical care provider at hospital 504 as the patient continues to recover at home. Container 502 includes the first electrical control circuitry 504 built into or received in a receptacle 506 in the container; the controllable lighting system 508 built into or received in a receptacle 510 in the container 502; at least one receptacle 512 adapted to receive at least one of the imaging system 514, the video output device 516, the audio input device 518, and the audio output device 520; and an outer shell 522 adapted to contain and protect the first electrical control circuitry 504, controllable lighting system 508, imaging system 514, video output device 516, audio input device 518, and audio output device 520 during transport. Controllable lighting system 508 may be mounted on a mounting structure 524, here depicted as a mechanical linkage. Mounting structure may be configured to be attached to and support by container 502, e.g., so it can be extended for use, and retracted in receptable 510 when it is not in use. In other aspects, the mounting structure may be a separate, self-supporting structure such as a tripod or pole mounted on a base. The mounting structure can be extendable and/or expandable from a compact configuration suitable for storage in container 502 to an extended or expanded use configuration. The container may be reusable or disposable. In an aspect, the container may be sterilizable. In an aspect, the container includes a delivery label providing information, for example, regarding an address to which the container should be shipped when it is no longer in use by the subject. For example, the container could be returned to the hospital, or to a service location at which it could be sterilized, tested, calibrated, refurbished, etc. prior to being provided to another patient. In the embodiment depicted in FIG. 5, imaging system 514, video output device 516, audio input device 518, and audio output device 520 are built into display 522, which is configured to communicate with electrical control circuitry 504. As can be seen, also contained within container 502 is diagnostic device 530, in this example a blood pressure cuff and associated blood pressure sensing circuitry 532 in receptacle 534.

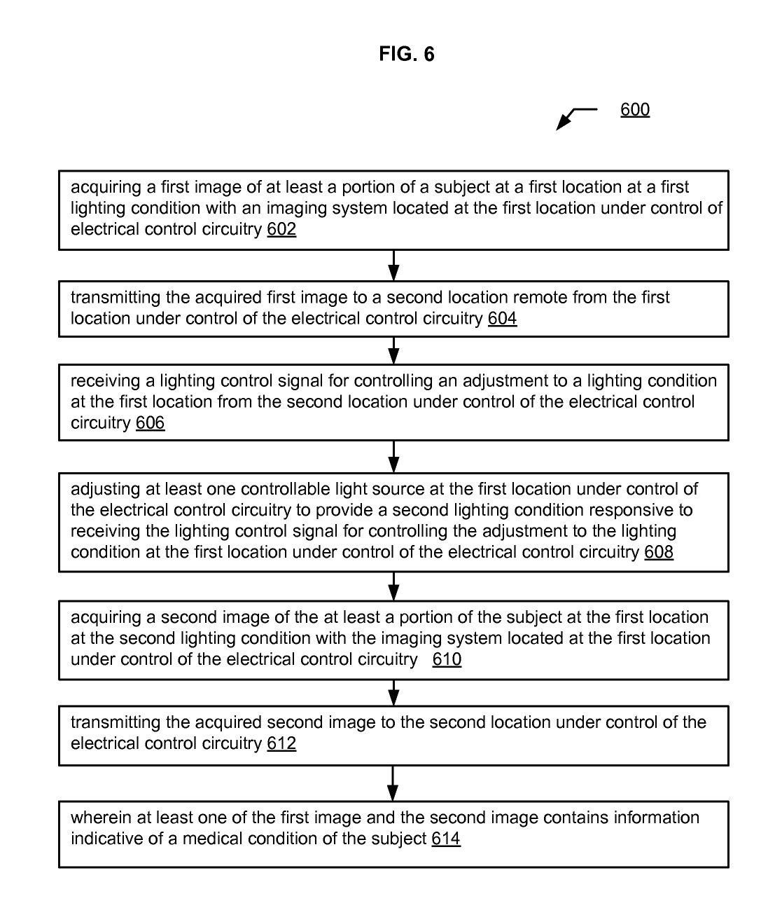

FIG. 6 shows a flow diagram outlining a method 600 of providing remote visualization of a subject. Method 600 includes acquiring a first image of at least a portion of a subject at a first location at a first lighting condition with an imaging system located at the first location under control of electrical control circuitry at 602; transmitting the acquired first image to a second location remote from the first location under control of the electrical control circuitry at 604; receiving a lighting control signal for controlling an adjustment to a lighting condition at the first location from the second location under control of the electrical control circuitry at 606; adjusting at least one controllable light source at the first location under control of the electrical control circuitry to provide a second lighting condition responsive to receiving the lighting control signal for controlling the adjustment to the lighting condition at the first location at 608; acquiring a second image of the at least a portion of the subject at the first location at the second lighting condition with the imaging system located at the first location under control of the electrical control circuitry at 610; and transmitting the acquired second image to the second location under control of the electrical control circuitry at 612. As indicated at 614, in an aspect, at least one of the first image and the second image contains information indicative of a health status of the subject. As described herein above, in an aspect the control signal generated at the remote location controls adjustment to the lighting system in order to increase the medical information content of the second image relative to the first image, as determined through image processing and analysis of the first image. Quality of the first image (e.g. medically relevant information content) may be determined through comparison of measured information (e.g. image features) to a standard (expected information content; e.g. has the desired level of contrast, resolution, focus, frequency content, level of illumination, etc. been obtained), and adjustment to one or more lighting parameters made if the image features fail to meet the standard. Alternatively, or in addition, the adjustment may be determined iteratively, by adjusting one or more lighting parameters, detecting an additional image, and determining the change in image quality (determined through analysis of one or more features of the image) relative to the previous image, until no further improvement in image quality can be obtained.

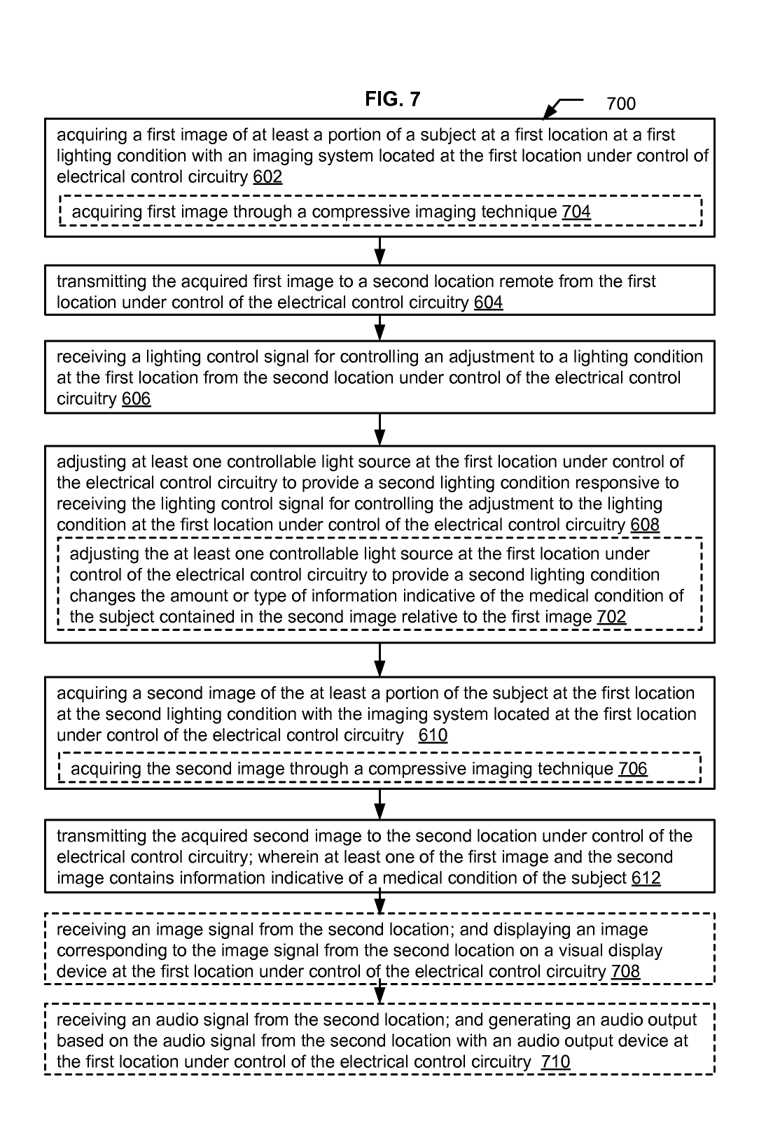

FIGS. 7-12 depict variations and expansions of method 600 as shown in FIG. 6. In the methods depicted in FIGS. 7-12, steps 602-614 are as described generally in connection with FIG. 6. Method steps outlined with dashed lines represent steps that are included in some, but not all method aspects, and combinations of steps other than those specifically depicted in the figures are possible as would be known by those having ordinary skill in the relevant art.

FIG. 7 depicts a method 700. In method 700, in an aspect, adjusting the at least one controllable light source at the first location under control of the electrical control circuitry to provide a second lighting condition changes the amount or type of information indicative of the health status of the subject contained in the second image relative to the first image, as indicated at 702.

In another aspect, method 700 includes acquiring at least one of the first image and the second image through a compressive imaging technique, as indicated at 704 and 706, respectively.

In an aspect, method 700 includes receiving an image signal from the second location and displaying an image corresponding to the image signal from the second location on a visual display device at the first location under control of the electrical control circuitry, as indicated at 708.

In another aspect, method 700 includes receiving an audio signal from the second location and generating an audio output based on the audio signal from the second location with an audio output device at the first location under control of the electrical control circuitry, as indicated at 710.

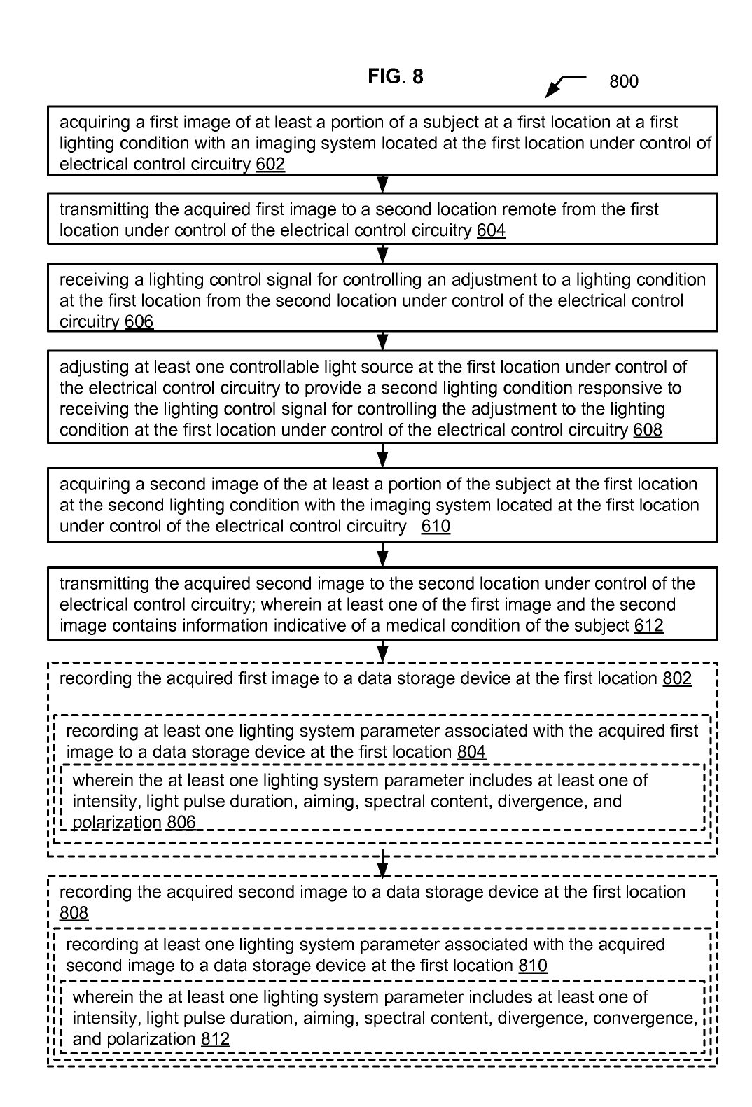

FIG. 8 depicts a method 800, which is a variant of method 600 shown in FIG. 6. Method 800 includes recording the acquired first image to a data storage device at the first location, as indicated at 802. Method 800 may also include recording at least one lighting system parameter associated with the acquired first image to a data storage device at the first location, as indicated at 804. The at least one lighting system parameter may include at least one of intensity, light pulse duration, aiming, spectral content, divergence or convergence, and polarization, as indicated at 806. Stored lighting system parameters associated with stored images may inform subsequent analysis. For example, an image acquired under a particular illumination wavelength may be expected to provide different information than an image acquired under a different illumination wavelength; thus two images acquired at different wavelengths may provide different, and complementary, information.

In another aspect, method 800 includes recording the acquired second image to a data storage device at the first location, as indicated at 808. In an aspect, method 800 includes recording at least one lighting system parameter associated with the acquired second image to a data storage device at the first location, as indicated at 810. In an aspect, the at least one lighting system parameter includes at least one of intensity, light pulse duration, aiming, spectral content, divergence, convergence, and polarization, as indicated at 812.

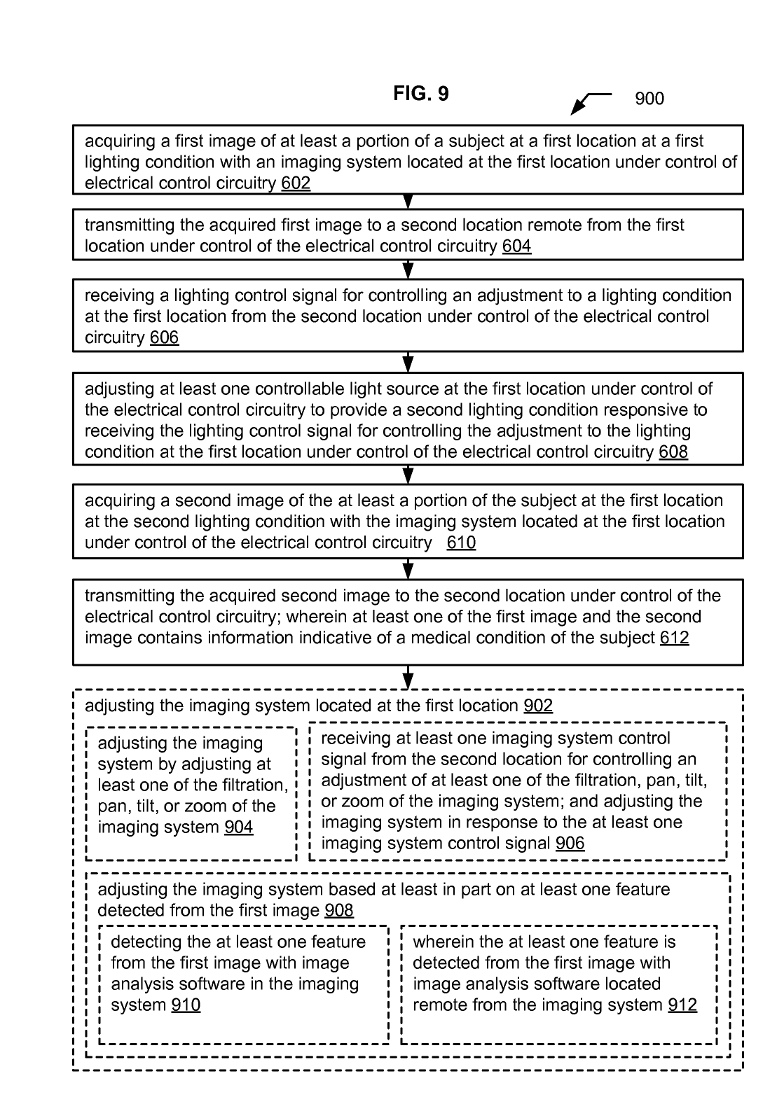

FIG. 9 shows a method 900 that is a further variant of method 600 shown in FIG. 6. In an aspect, as indicated at 902, method 900 includes adjusting the imaging system located at the first location, for example by adjusting at least one of the filtration, pan, tilt, or zoom of the imaging system, as indicated at 904. Adjusting filtration of the imaging system may be used to obtain images at particular light wavelengths or polarizations. Adjusting pan, tilt, or zoom may be used to selectively image particular portions of the subject, e.g. to image only portions of the subject that are of interest while excluding portions that are not of interest, or to obtain an image that provides greater detail of a particular portion of the subject.

In an aspect, a method 900 includes receiving at least one imaging system control signal from the second location for controlling an adjustment of at least one of the filtration, pan, tilt, or zoom of the imaging system, and adjusting the imaging system in response to the at least one imaging system control signal, as indicated at 906.

In another aspect, method 900 includes adjusting the imaging system based at least in part on at least one feature detected from the first image, at 908. In an aspect, method 900 includes detecting the at least one feature from the first image with image analysis software in the imaging system, as indicated at 910. In another aspect, the at least one feature is detected from the first image with image analysis software located remote from the imaging system, as indicated at 912.

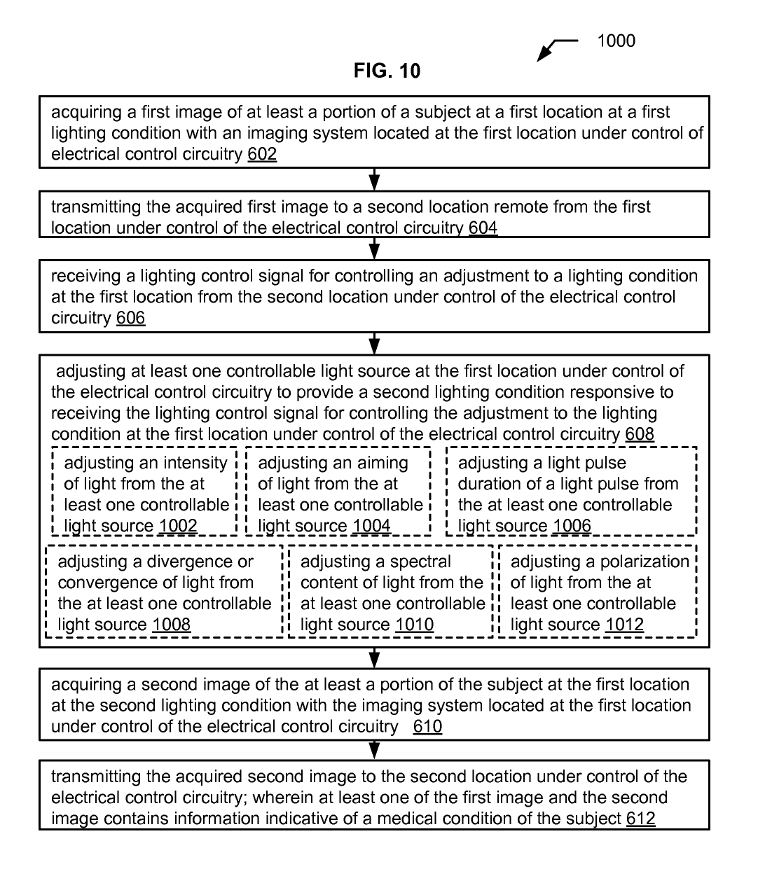

As shown in FIG. 10, in an aspect, a method 1000 includes adjusting an intensity of light from the at least one controllable light source, as indicated at 1002; adjusting an aiming of light from the at least one controllable light source, as indicated at 1004; adjusting a light pulse duration of a light pulse from the at least one controllable light source, as indicated at 1006; adjusting a divergence or convergence of light from the at least one controllable light source, as indicated at 1008; adjusting a spectral content of light from the at least one controllable light source, as indicated at 1010; or adjusting a polarization of light from the at least one controllable light source, as indicated at 1012. The intensity of light from a controllable light source can be adjusted by adjusting the intensity of light generated by the light source (e.g. by adjusting the current driving the light source), or by using a controllable filter or shutter to reduce the intensity of light generated by the light source. Aiming of light from the light source can be accomplished by using a reflector to direct the light from the light source, or by adjusting the position of the light source itself, either of which can be accomplished with mechanical linkages, as discussed herein above. Furthermore, in an aspect the position of the light source can be modified by selectively activating different individual light sources having different positions or orientations within a compound light source. Adjusting the duration of a light pulse can be accomplished through the use of appropriately configured circuitry driving generation of light by the light source, or by the use of a controllable filter or shutter to selectively block the light. Divergence or convergence of light from the controllable light source can be adjusted through the use of a controllable lens. Adjusting divergence or convergence of light is contemplated to include reducing or increasing divergence or convergence of light. As discussed herein above, adjusting spectral content or polarization of light from the controllable light source can be accomplished through the use of a controllable light, a controllable filter, or selective activation of multiple light sources having different spectral and/or polarization properties.

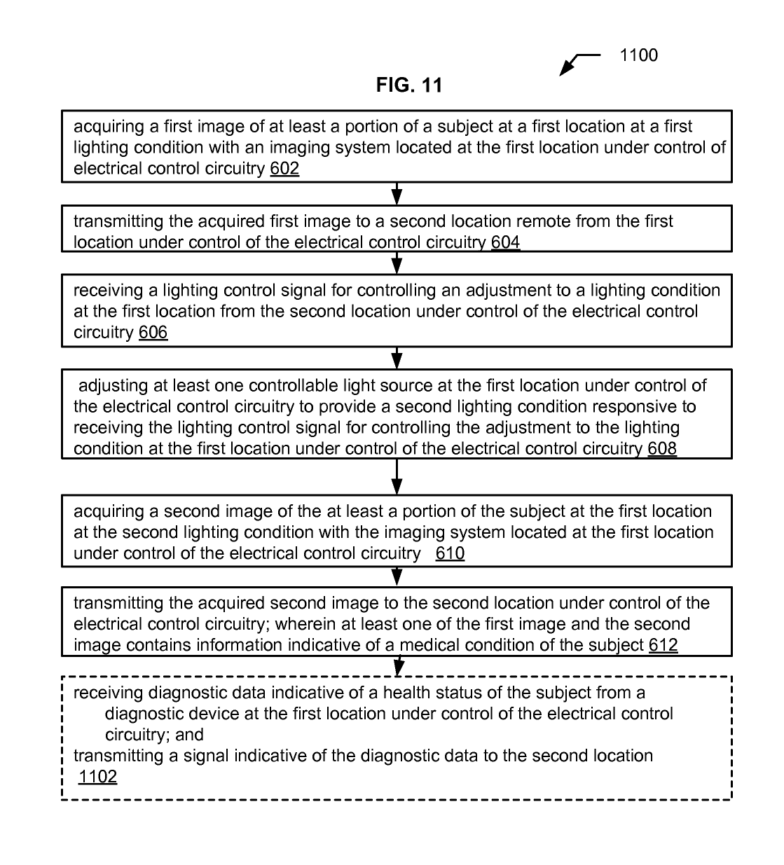

As shown in FIG. 11, in an aspect, a method 1100 includes receiving diagnostic data indicative of a health status of the subject from a diagnostic device at the first location under control of the electrical control circuitry; and transmitting a signal indicative of the diagnostic data to the second location, as indicated at 1102.

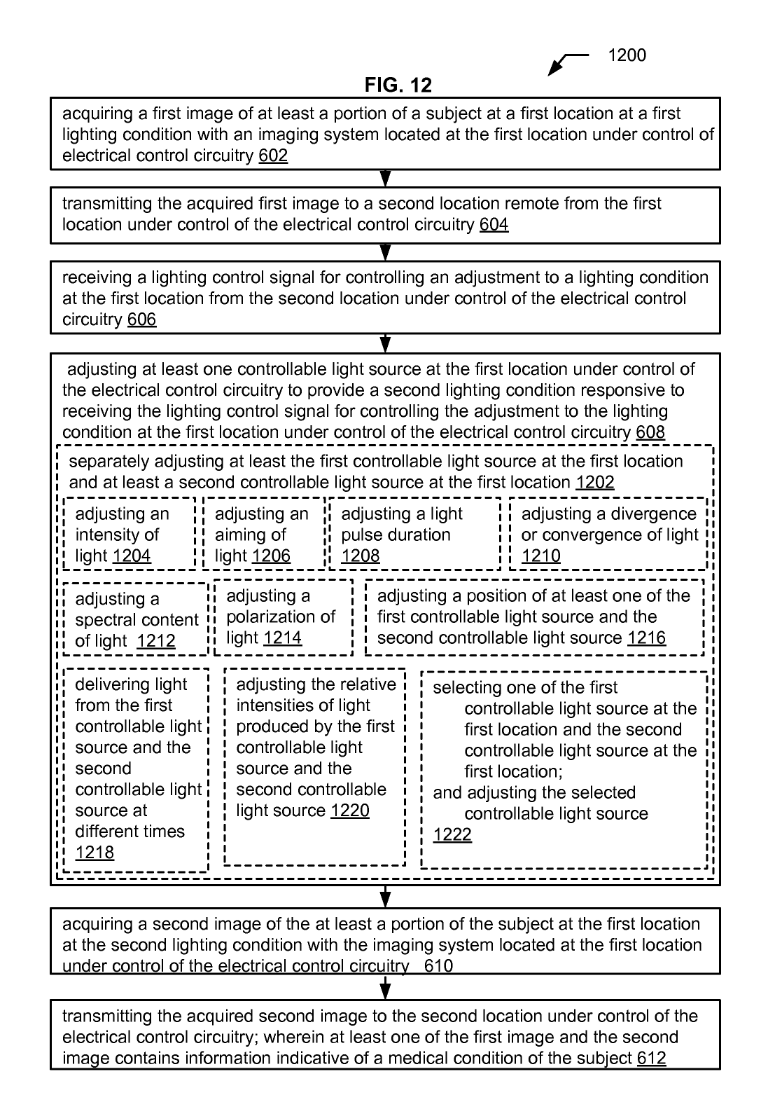

In another aspect, in a method 1200 as shown in FIG. 12, adjusting the at least one controllable light source includes separately adjusting at least a first controllable light source at the first location and at least a second controllable light source at the first location, as indicated at 1202. Controlling at least one of the controllable light sources, e.g. at least the first controllable light source or second controllable light source may include adjusting an intensity (at 1204), aiming (at 1206), light pulse duration (at 1208), divergence or convergence (at 1210), spectral content (at 1212), or polarization of light (at 1214) from at least one of the first controllable light source or the second controllable light source, or adjusting a position of at least one of the first controllable light source or the second controllable light source (at 1216).

As discussed herein above, adjusting intensity, aiming, light pulse duration, divergence or convergence, spectral content, or polarization of light from the first controllable light source may involve controlling the generation of light by the light source, or controlling a filter, shutter, mirror, etc. that modify the light from the light source without modifying the operation of the light source itself

In another aspect, separately adjusting at least the first controllable light source at the first location and at least the second controllable light source at the first location includes delivering light from the first controllable light source and the second controllable light source at different times, as indicated at 1218. In another aspect, separately adjusting at least the first controllable light source at the first location and at least the second controllable light source at the first location includes adjusting the relative intensities of light produced by the first controllable light source and the second controllable light source, as indicated at 1220. In an aspect, separately adjusting at least the first controllable light source and the second controllable light includes selecting one of the first controllable light source at the first location and the second controllable light source at the first location, and adjusting the selected controllable light source 1222. The selected light source can be selected in order to control the spectral content, polarization, aiming, position, or other parameter of light produced by the light source.

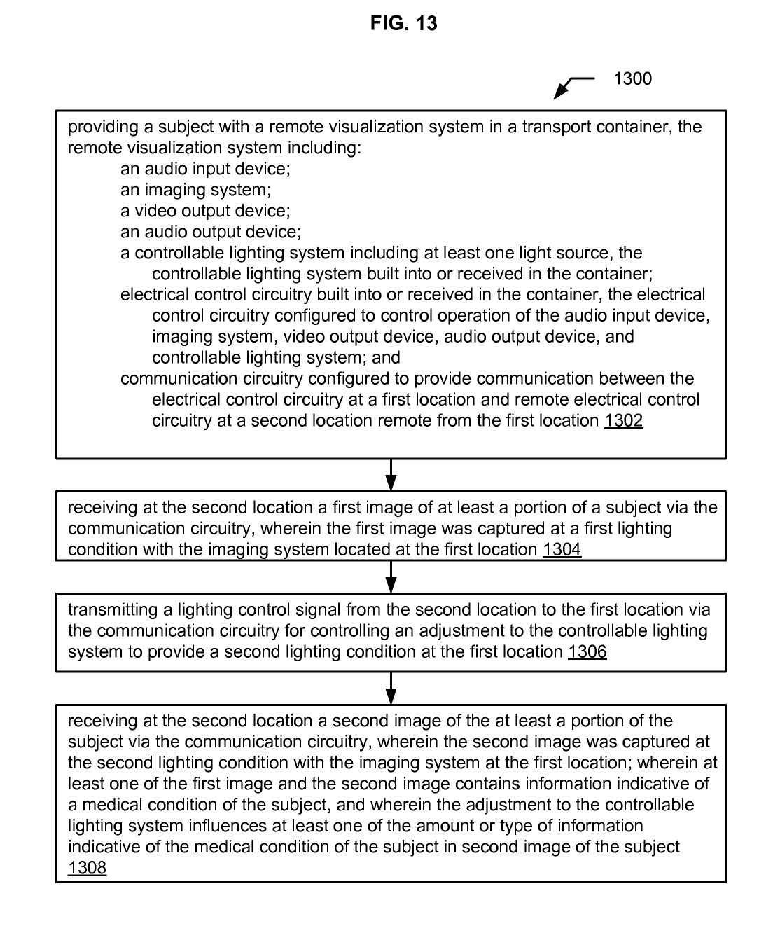

FIG. 13 illustrates a further method 1300 of providing remote visualization of a subject. In an aspect, method 1300 is implemented at a medical monitoring location, e.g. second location 108 as shown in FIG. 1 or location 208 in FIG. 2, and using a medical monitoring system, e.g. system 106 in the embodiment of FIG. 1 or system 206 in FIG. 2. Method 1300 includes providing a subject with a remote visualization system in a transport container, at 1302. The remote visualization system is generally as described herein above, and includes an audio input device; an imaging system; a video output device; an audio output device; a controllable lighting system including at least one light source, the controllable lighting system built into or received in the container; electrical control circuitry built into or received in the container, the electrical control circuitry configured to control operation of the audio input device, imaging system, video output device, audio output device, and controllable lighting system; and communication circuitry configured to provide communication between the electrical control circuitry at a first location and remote electrical control circuitry at a second location remote from the first location.

Method 1300 further includes receiving at the second location a first image of at least a portion of a subject via the communication circuitry, wherein the first image was captured at a first lighting condition with the imaging system located at the first location, at 1304; transmitting a lighting control signal from the second location to the first location via the communication circuitry for controlling an adjustment to the controllable lighting system to provide a second lighting condition at the first location at 1306; and receiving at the second location a second image of the at least a portion of the subject via the communication circuitry, wherein the second image was captured at the second lighting condition with the imaging system at the first location; wherein at least one of the first image and the second image contains information indicative of a health status of the subject, and wherein the adjustment to the controllable lighting system influences at least one of the amount or type of information indicative of the health status of the subject in the second image of the subject, as indicated at 1308.

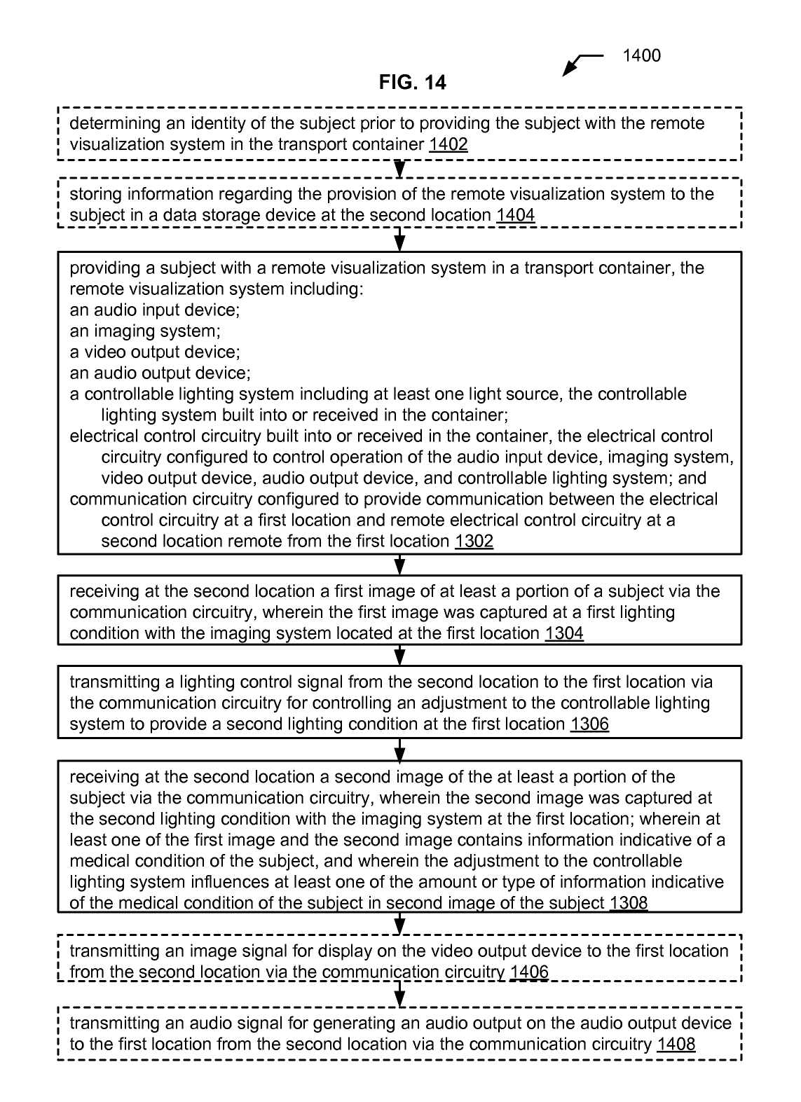

FIGS. 14-18 depict variations and expansions of method 1300 as shown in FIG. 13. In the methods depicted in FIGS. 14-18, steps 1302-1308 are as described generally in connection with FIG. 13. In FIG. 14, in an aspect, a method 1400 includes determining an identity of the subject prior to providing the subject with the remote visualization system in the transport container, as indicated at 1402. For example, identity of the subject may be based on name, patient identification number, insurance identification number, or the like. Subject identity may be used to link images and other data obtained with the remote visualization system with other patient medical records, for example. In various aspects, a method includes storing information regarding the provision of the remote visualization system to the subject in a data storage device at the second location, as indicated at 1404. Storing information regarding the provision of the remote visualization system to the subject may include, for example, storing information regarding one or more of the identity of the patient; identity of a medical care provider or caregiver associated with the patient; identity of at least a portion of the remote visualization system (e.g. serial number or other identifying number, equipment type, components, settings, or configuration); time, date or location at which the remote visualization system was provided to the subject; or time, date, or location at which the remote visualization system should be returned by the subject, for example. In additional aspects, method 1400 includes transmitting an image signal for display on the video output device to the first location from the second location via the communication circuitry, as indicated at 1406, or transmitting an audio signal for generating an audio output on the audio output device to the first location from the second location via the communication circuitry, as indicated at 1408.

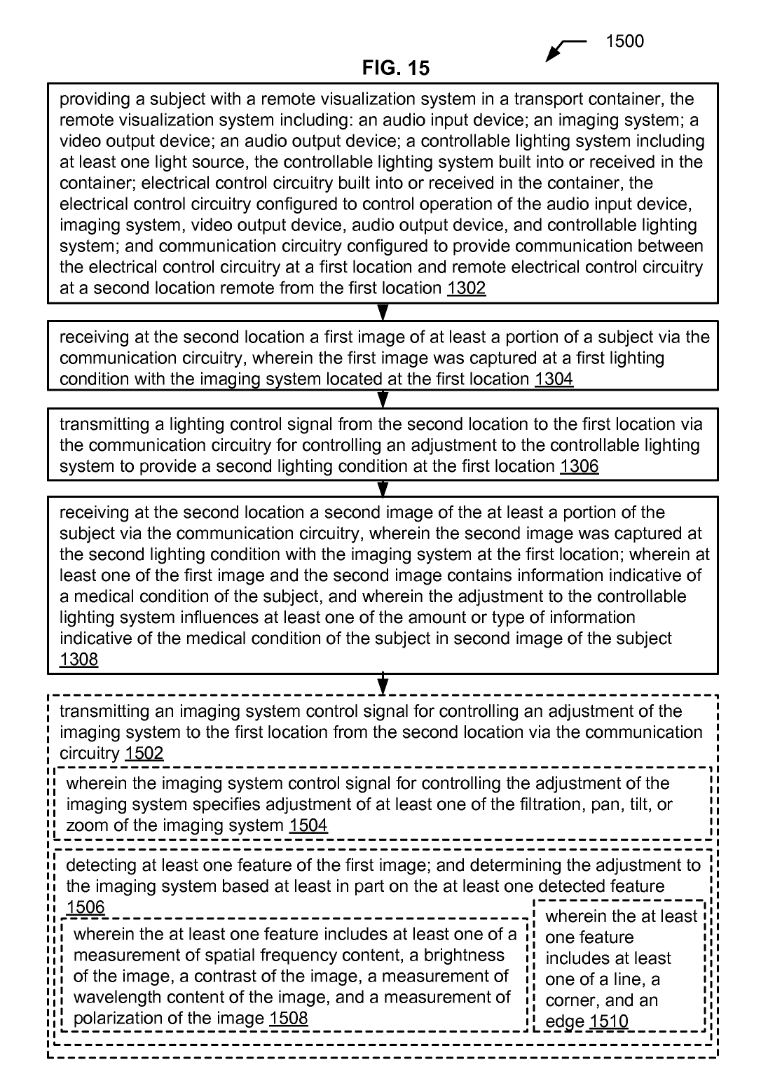

As shown in FIG. 15, in an aspect, a method 1500 includes transmitting an imaging system control signal for controlling an adjustment of the imaging system to the first location from the second location via the communication circuitry, as indicated at 1502. The imaging system control signal for controlling the adjustment of the imaging system may specify adjustment of at least one of the filtration, pan, tilt, or zoom of the imaging system, as indicated at 1504.

In an aspect, method 1500 includes detecting at least one feature of the first image and determining the adjustment to the imaging system based at least in part on the at least one detected feature, as indicated at 1506. In various aspects, the at least one feature includes at least one of a measurement of spatial frequency content, a brightness of the image, a contrast of the image, a measurement of wavelength content of the image, and a measurement of polarization of the image, as indicated at 1508, or at least one of a line, a corner, and an edge, as indicated at 1510.

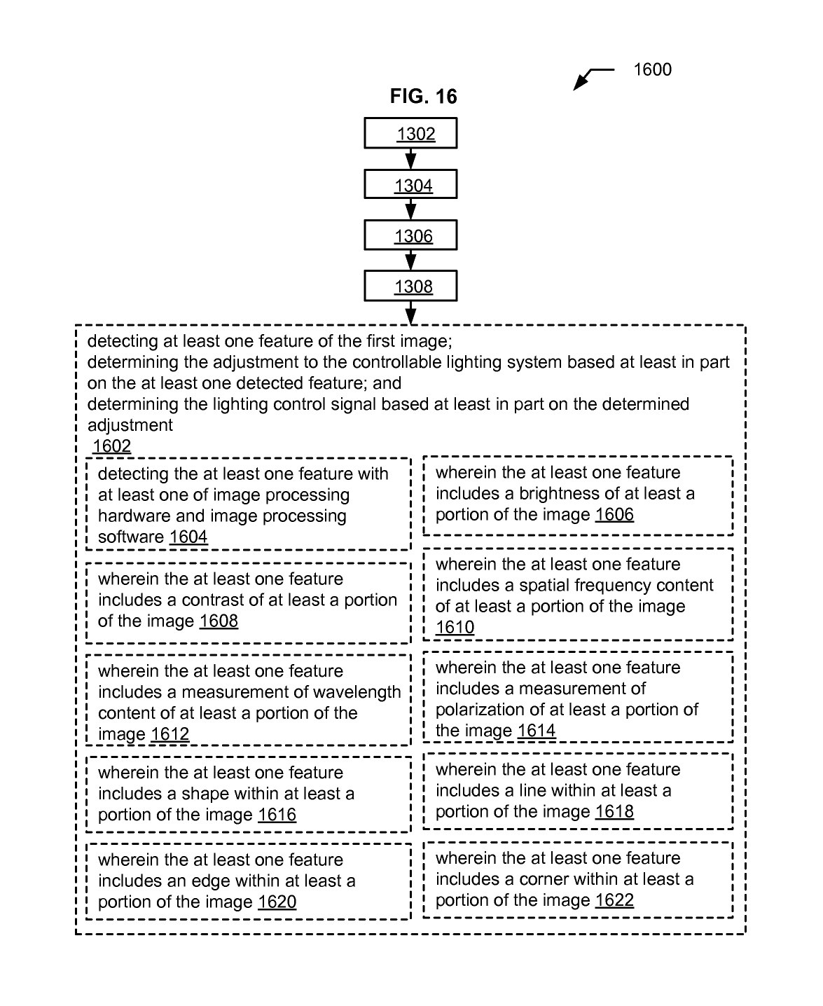

As shown in FIG. 16, in an aspect, a method 1600 includes detecting at least one feature of the first image, determining the adjustment to the controllable lighting system based at least in part on the at least one detected feature, and determining the lighting control signal based at least in part on the determined adjustment, as indicated at 1602. Method 1600 may include detecting the at least one feature with at least one of image processing hardware and image processing software, as indicated at 1604.

In an aspect, the at least one feature may include at least one of brightness (at 1606), contrast (at 1608), spatial frequency content (at 1610), a measurement of wavelength content (at 1612), or a measurement of polarization (at 1614) of at least a portion of the image. In addition, the at least one feature may include a shape (at 1616), line (at 1618), edge (at 1620), or corner (at 1622) within at least a portion of the image.

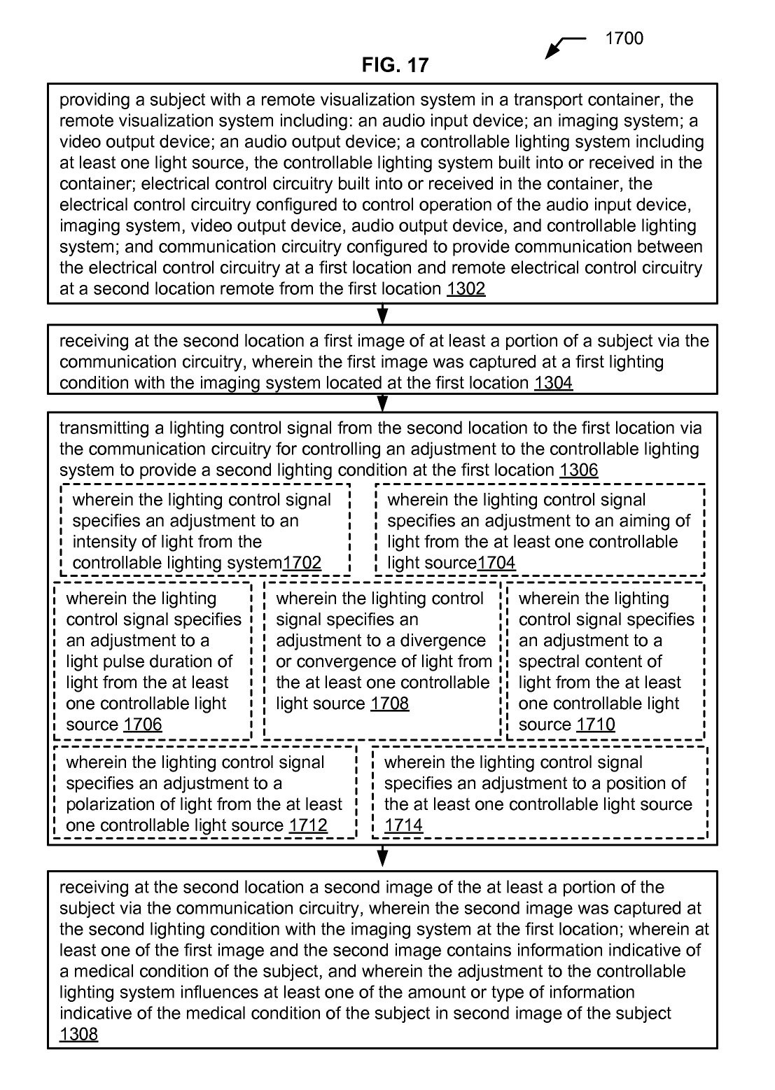

FIG. 17 depicts a method 1700 showing method variants relating to the lighting control signal. In various aspects, the lighting control signal specifies an adjustment to at least one of intensity (at 1702), aiming (at 1704), light pulse duration (at 1706), divergence or convergence (at 1708), spectral content (at 1710), and polarization (at 1712) of light from the at least one controllable light source, and adjustment to a position of the at least one controllable light source (at 1714).

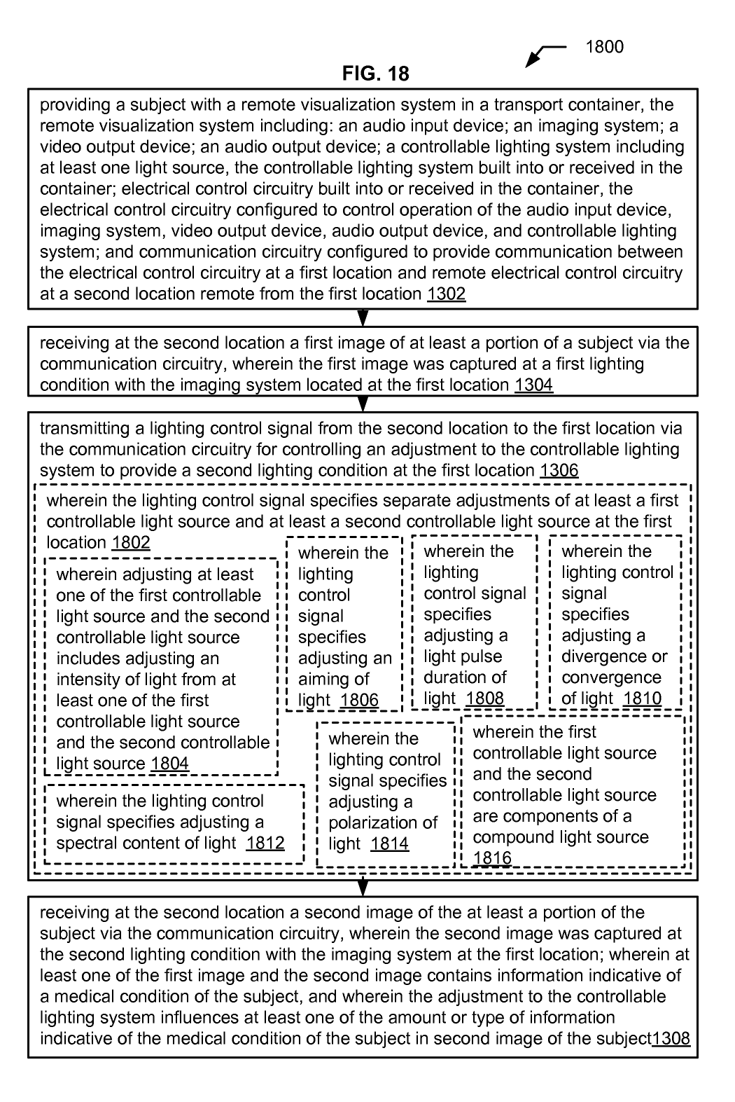

As shown in FIG. 18, in an aspect of method 1800, the lighting control signal specifies separate adjustments of at least a first controllable light source and at least a second controllable light source at the first location, as indicated at 1802. In various aspects, the lighting control signal specifies adjusting at least one of an intensity of light from at least one of the first controllable light source and the second controllable light source, as indicated at 1804; adjusting an aiming of light from at least one of the first controllable light source and the second controllable light source, as indicated at 1806; adjusting a light pulse duration of light from at least one of the first controllable light source and the second controllable light source, as indicated at 1808; adjusting a divergence or convergence of light from at least one of the first controllable light source and the second controllable light source, as indicated at 1810; adjusting a spectral content of light from at least one of the first controllable light source and the second controllable light source, as indicated at 1812; and adjusting a polarization of light from at least one of the first controllable light source and the second controllable light source, as indicated at 1814. In an aspect, the first controllable light source and the second controllable light source are components of a compound light source, as indicated at 1816.

In various embodiments, methods as described herein may be performed according to instructions implementable in hardware, software, and/or firmware. Such instructions may be stored in non-transitory machine-readable data storage media, for example. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware in one or more machines, compositions of matter, and articles of manufacture. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures. Electrical circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components.

Implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled/ /implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to non-transitory machine-readable data storage media such as a recordable type medium such as a floppy disk, a hard disk drive, a Compact Disc (CD), a Digital Video Disk (DVD), a digital tape, a computer memory, etc.. A signal bearing medium may also include transmission type medium such as a digital and/or an analog communication medium (e.g., a fiber optic cable, a waveguide, a wired communications link, a wireless communication link (e.g., transmitter, receiver, transmission logic, reception logic, etc.) and so forth).

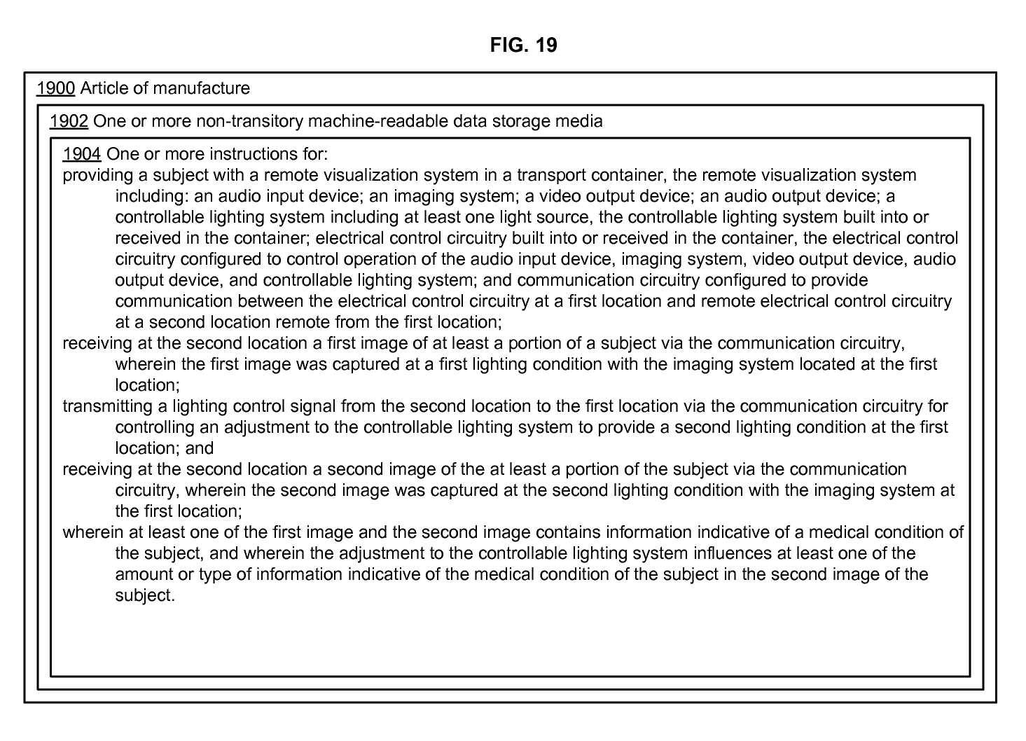

FIG. 19 depicts an article of manufacture 1900 that includes one or more non-transitory machine-readable data storage media 1902 bearing one or more instructions 1904 for: providing a subject with a remote visualization system in a transport container, the remote visualization system including an audio input device, an imaging system, a video output device, an audio output device, a controllable lighting system including at least one light source, the controllable lighting system built into or received in the container, electrical control circuitry built into or received in the container, the electrical control circuitry configured to control operation of the audio input device, imaging system, video output device, audio output device, and controllable lighting system, and communication circuitry configured to provide communication between the electrical control circuitry at a first location and remote electrical control circuitry at a second location remote from the first location; receiving at the second location a first image of at least a portion of a subject via the communication circuitry, wherein the first image was captured at a first lighting condition with the imaging system located at the first location; transmitting a lighting control signal from the second location to the first location via the communication circuitry for controlling an adjustment to the controllable lighting system to provide a second lighting condition at the first location; and receiving at the second location a second image of the at least a portion of the subject via the communication circuitry, wherein the second image was captured at the second lighting condition with the imaging system at the first location; wherein at least one of the first image and the second image contains information indicative of a health status of the subject, and wherein the adjustment to the controllable lighting system influences at least one of the amount or type of information indicative of the health status of the subject in the second image of the subject. Instructions 1904 depicted in FIG. 19 corresponds to method 1300 shown in FIG. 13. Other variants of methods as depicted in FIGS. 13-18 and as described herein can be implemented through the use of non-transitory machine-readable data storage media bearing one or more suitable instructions.

For example, and without limitation, in an aspect, the one or more non-transitory machine-readable data storage media 1902 bear one or more instructions for storing information regarding the provision of the remote visualization system to the subject in a data storage device at the first location. In aspect, the one or more non-transitory machine-readable data storage media 1902 bear one or more instructions for transmitting an imaging system control signal for controlling an adjustment of the imaging system to the first location from the second location via the communication circuitry. For example, the one or more non-transitory machine-readable data storage media may bear one or more instructions for generating the imaging system control signal to control adjustment of the imaging system by adjusting at least one of the filtration, pan, tilt, or zoom of the imaging system. Alternatively, or in addition, the one or more non-transitory machine-readable data storage media 1902 may bear one or more instructions for detecting at least one feature of the first image; and determining the adjustment to the imaging system based at least in part on the at least one detected feature. In some aspects, the one or more non-transitory machine-readable data storage media 1902 bear one or more instructions for: detecting at least one feature of the first image; determining the adjustment to the controllable lighting system based at least in part on the at least one detected feature; and determining the lighting control signal based at least in part on the determined adjustment.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of providing remote visualization of a subject comprising:
acquiring a first image of at least a portion of a subject at a first location at a first lighting condition with an imaging system located at the first location under control of electrical control circuitry;
transmitting the acquired first image to a second location remote from the first location under control of the electrical control circuitry;
receiving a lighting control signal for controlling an adjustment to a lighting condition at the first location from the second location under control of the electrical control circuitry;
adjusting at least one controllable light source at the first location under control of the electrical control circuitry to provide a second lighting condition responsive to receiving the lighting control signal for controlling the adjustment to the lighting condition at the first location;
acquiring a second image of the at least a portion of the subject at the first location at the second lighting condition with the imaging system located at the first location under control of the electrical control circuitry; and
transmitting the acquired second image to the second location under control of the electrical control circuitry;
wherein at least one of the first image and the second image contains information indicative of a health status of the subject.

2. The method of claim 1, wherein adjusting the at least one controllable light source at the first location under control of the electrical control circuitry to provide the second lighting condition changes the amount or type of information indicative of the health status of the subject contained in the second image relative to the first image.

3. The method of claim 1, comprising:
acquiring at least one of the first image and the second image through a compressive imaging technique.

4.-8. (canceled)

9. The method of claim 1, comprising:
recording the acquired second image to a data storage device at the first location.

10.-11. (canceled)

12. The method of claim 1, comprising:
adjusting the imaging system located at the first location.

13. (canceled)

14. The method of claim 12, comprising:
receiving at least one imaging system control signal from the second location for controlling an adjustment of at least one of the filtration, pan, tilt, or zoom of the imaging system; and
adjusting the imaging system in response to the at least one imaging system control signal.

15. The method of claim 12, comprising:
adjusting the imaging system based at least in part on at least one feature detected from the first image.

16.-23. (canceled)

24. The method of claim 1, including separately adjusting at least the first controllable light source at the first location and at least a second controllable light source at the first location.

25.-35. (canceled)

36. A remote visualization system comprising:
an audio input device at a first location;
an imaging system at the first location adapted to acquire an image of a subject, the image containing information indicative of a health status of the subject;
a video output device at the first location;
an audio output device at the first location;
a controllable lighting system including at least one light source adapted to illuminate at least a portion of the subject during acquisition of the image of the subject and having at least one controllable lighting system parameter that influences at least one of the amount or type of information indicative of the health status of the subject in an acquired image of the subject;
first electrical control circuitry at the first location operatively connected to and configured to control operation of the audio input device, imaging system, video output device, audio output device, and controllable lighting system; and
communication circuitry at the first location configured to provide communication between the first electrical control circuitry and second electrical control circuitry at a second location remote from the first location;
wherein the first electrical control circuitry is configured to control the controllable parameter of the controllable lighting system responsive to receipt of a lighting control signal from the second electrical control circuitry at the second location.

37. The remote visualization system of claim 36, wherein the controllable lighting system is adapted to generate a light pulse having a pulse duration.

38.-84. (canceled)

85. The remote visualization system of claim 36, wherein the communication circuitry is configured to provide communication with the second electrical control circuitry at the second location via a wireless communication link.

86.-98. (canceled)

99. The remote visualization system of claim 36, comprising:
a mounting structure configured to support at least one of the controllable lighting system, the imaging system, the video output device, the audio input device, and the audio output device.

100.-101. (canceled)

102. The remote visualization system of claim 36, comprising a container, the container including:
the first electrical control circuitry built into or received in a receptacle in the container;
the controllable lighting system built into or received in a receptacle in the container;
at least one receptacle adapted to receive at least one of the imaging system, the video output device, the audio input device, and the audio output device; and
an outer shell adapted to contain and protect the first electrical control circuitry, controllable lighting system, and the at least one of the imaging system, the video output device, the audio input device, and the audio output device during transport.

103.-106. (canceled)

107. A method of providing remote visualization of a subject comprising:
providing a subject with a remote visualization system in a transport container, the remote visualization system including:
an audio input device;
an imaging system;
a video output device;
an audio output device;
a controllable lighting system including at least one light source, the controllable lighting system built into or received in the container;
electrical control circuitry built into or received in the container, the electrical control circuitry configured to control operation of the audio input device, imaging system, video output device, audio output device, and controllable lighting system; and
communication circuitry configured to provide communication between the electrical control circuitry at a first location and remote electrical control circuitry at a second location remote from the first location;
receiving at the second location a first image of at least a portion of a subject via the communication circuitry, wherein the first image was captured at a first lighting condition with the imaging system located at the first location;
transmitting a lighting control signal from the second location to the first location via the communication circuitry for controlling an adjustment to the controllable lighting system to provide a second lighting condition at the first location; and
receiving at the second location a second image of the at least a portion of the subject via the communication circuitry, wherein the second image was captured at the second lighting condition with the imaging system at the first location;
wherein at least one of the first image and the second image contains information indicative of a health status of the subject, and wherein the adjustment to the controllable lighting system influences at least one of the amount or type of information indicative of the health status of the subject in the second image of the subject.

108.-111. (canceled)

112. The method of claim 107, comprising:
transmitting an imaging system control signal for controlling an adjustment of the imaging system to the first location from the second location via the communication circuitry.

113. (canceled)

114. The method of claim 112, comprising:
detecting at least one feature of the first image; and
determining the adjustment to the imaging system based at least in part on the at least one detected feature.

115.-116. (canceled)

117. The method of claim 107, comprising:
detecting at least one feature of the first image;
determining the adjustment to the controllable lighting system based at least in part on the at least one detected feature; and
determining the lighting control signal based at least in part on the determined adjustment.

118.-134. (canceled)

135. The method of claim 107, wherein the lighting control signal specifies separate adjustments of at least a first controllable light source and at least a second controllable light source at the first location.

136.-142. (canceled)

143. An article of manufacture comprising:
one or more non-transitory machine-readable data storage media bearing one or more instructions for:
providing a subject with a remote visualization system in a transport container, the remote visualization system including:
an audio input device;
an imaging system;
a video output device;
an audio output device;
a controllable lighting system including at least one light source, the controllable lighting system built into or received in the container;
electrical control circuitry built into or received in the container, the electrical control circuitry configured to control operation of the audio input device, imaging system, video output device, audio output device, and controllable lighting system; and
communication circuitry configured to provide communication between the electrical control circuitry at a first location and remote electrical control circuitry at a second location remote from the first location;
receiving a first image of at least a portion of a subject at the second location via the communication circuitry, wherein the first image was captured at a first lighting condition with the imaging system located at the first location;
transmitting a lighting control signal from the second location to the first location via the communication circuitry for controlling an adjustment to the controllable lighting system to provide a second lighting condition at the first location; and
receiving at the second location a second image of the at least a portion of the subject via the communication circuitry, wherein the second image was captured at the second lighting condition with the imaging system at the first location;
wherein at least one of the first image and the second image contains information indicative of a health status of the subject, and wherein the adjustment to the controllable lighting system influences at least one of the amount or type of information indicative of the health status of the subject in the second image of the subject.

144.-149. (canceled)

150. The method of claim 1, comprising at least one of:
receiving an image signal from the second location, and displaying an image corresponding to the image signal from the second location on a visual display device at the first location under control of the electrical control circuitry; and
receiving an audio signal from the second location, and generating an audio output based on the audio signal from the second location with an audio output device at the first location under control of the electrical control circuitry.

151. The method of claim 1, wherein adjusting the at least one controllable light source includes adjusting at least one of an intensity of light, an aiming of light, a light pulse duration of a light pulse, a divergence or convergence of light, a spectral content of light, and a polarization of light from the at least one controllable light source.

152. The method of claim 24, wherein separately adjusting at least the first controllable light source at the first location and at least the second controllable light source at the first location includes at least one of:
adjusting an intensity of light from at least one of the first controllable light source and the second controllable light source;
adjusting an aiming of light from at least one of the first controllable light source and the second controllable light source;
adjusting a position of at least one of the first controllable light source and the second controllable light source;
adjusting a light pulse duration of light from at least one of the first controllable light source and the second controllable light source;
adjusting a divergence or convergence of light from at least one of the first controllable light source and the second controllable light source;
adjusting a spectral content of light from at least one of the first controllable light source and the second controllable light source;
adjusting a polarization of light from at least one of the first controllable light source and the second controllable light source;
delivering light from the first controllable light source and the second controllable light source at different times;
adjusting the relative intensities of light produced by the first controllable light source and the second controllable light source; and
selecting one of the first controllable light source at the first location and the second controllable light source at the first location and adjusting the selected controllable light source.

153. The remote visualization system of claim 37, wherein the first electrical control circuitry is configured to control generation of the light pulse by the controllable lighting system, and wherein the first electrical control circuitry is configured to coordinate detection of an image by the imaging system with generation of the light pulse by the controllable lighting system.

154. The remote visualization system of claim 36, wherein the controllable lighting system includes at least one of:
at least a first light source and a second light source, wherein the first light source differs from the second light source with regard to at least one of position, waveband, and polarization of light produced; and
at least one compound light source, the compound light source including at least two individual light sources.

155. The remote visualization system of claim 36, wherein the controllable lighting system includes at least one of an ultraviolet light source, an infrared light source, a visible light source, and a long-wave infrared light source.

156. The remote visualization system of claim 36, wherein the controllable lighting system includes at least one of a controllable optical system, a reflector, a filter, a lens, and a controllable positioning system.

157. The remote visualization system of claim 36, wherein the at least one light source has at least one of a controllable intensity, a controllable spectral content, and a controllable polarization.

158. The remote visualization system of claim 36, wherein at least a portion of the remote visualization system is configured as at least one of a hand-held unit, a mobile robot, a smart phone and a tablet computer.

159. The remote visualization system of claim 36, comprising at least one of a diagnostic device, a treatment delivery device, and a substance delivery device.

160. The remote visualization system of claim 36, comprising at least one diagnostic device, the diagnostic device including at least one of a blood pressure cuff, a thermometer, a stethoscope, an ECG monitor, an EEG monitor, a bioelectromagnetic sensor, an ultrasound probe, a chemical sensor, a gas sensor, a touch probe, and a bed mat sensor.

161. The remote visualization system of claim 36, comprising at least one substance delivery device, the substance delivery device including at least one of a controllable medication dispensing device configured to dispense at least one formulated medication in response to a control signal from the first electrical control circuitry, a transdermal substance delivery device configured to deliver substance to the subject in response to a control signal from the first electrical control circuitry, and an infusion system configured to deliver an infusible substance in response to a control signal from the first electrical control circuitry.

162. The remote visualization system of claim 36, wherein the imaging system includes at least one of a photocell, a charge-coupled device, a scanner, a 3D scanner, a 3D imager, a camera, a single pixel camera, a visual camera, an IR camera, a stereoscopic camera, a digital camera, a video camera, or a high speed video camera.

163. The remote visualization system of claim 102, wherein the container is at least one of a reusable container, a sterilizable container, a disposable container, and a container including a delivery label.

164. The method of claim 107, comprising at least one of:
determining an identity of the subject prior to providing the subject with the remote visualization system in the transport container;
storing information regarding the provision of the remote visualization system to the subject in a data storage device at the second location;
transmitting an image signal for display on the video output device to the first location from the second location via the communication circuitry; and
transmitting an audio signal for generating an audio output on the audio output device to the first location from the second location via the communication circuitry.

165. The method of claim 117, wherein the at least one feature includes at least one of a brightness of at least a portion of the image, a contrast of at least a portion of the image, a spatial frequency content of at least a portion of the image, a shape within at least a portion of the image, a line within at least a portion of the image, an edge within at least a portion of the image, a corner within at least a portion of the image, a measurement of wavelength content of at least a portion of the image, and a measurement of polarization of at least a portion of the image.

166. The method of claim 107, wherein the lighting control signal specifies at least one of an adjustment to an intensity of light from the controllable lighting system, an adjustment to an aiming of light from the at least one controllable light source, an adjustment to a position of the at least one controllable light source, an adjustment to a light pulse duration of light from the at least one controllable light source, an adjustment to a divergence or convergence of light from the at least one controllable light source, an adjustment to a spectral content of light from the at least one controllable light source, and an adjustment to a polarization of light from the at least one controllable light source.

167. The method of claim 135, wherein the lighting control signal specifies at least one of adjusting an intensity of light from at least one of the first controllable light source and the second controllable light source, adjusting an aiming of light from at least one of the first controllable light source and the second controllable light source, adjusting a light pulse duration of light from at least one of the first controllable light source and the second controllable light source, adjusting a divergence or convergence of light from at least one of the first controllable light source and the second controllable light source, adjusting a spectral content of light from at least one of the first controllable light source and the second controllable light source, and adjusting a polarization of light from at least one of the first controllable light source and the second controllable light source.
